(19) United States
(12) Patent Application Publication
(10) Pub. No.: US 2004/0229341 A1
(43) Pub. Date: Nov. 18, 2004

(54) Method for the production of chitin deacetylase

(76) Inventors: Marie-France Versali, Tilff (BE); Clerisse Fabienne, Braives (BE); Carole Gauthier, Liege (BE); Francoise Dumont, Grivegnee (BE); Jacques Dommes, La Reid (BE)

Correspondence Address: KNOBBE MARTENS OLSON & BEAR LLP, 2040 MAIN STREET, FOURTEENTH FLOOR, IRVINE, CA, 92614, US

(21) Appl. No.: 10/777,460

(22) Filed: Feb. 12, 2004

Related U.S. Application Data
(60) Provisional application No. 60/447,047, filed on Feb. 12, 2003.

(51) Int. Cl. C12N 1/16; C12N 9/18; C08B 37/08; C07H 21/04; C12N 15/74

(52) U.S. Cl. 435/254.3; 435/69.1; 435/197; 435/320.1; 536/20; 536/23.2

(57) ABSTRACT

In a first aspect, the present invention relates to a recombinant fungal strain, capable of expressing chitin deacetylase, which includes an expression vector that contains a nucleic acid molecule encoding chitin deacetylase, a suitable promoter and a transcription terminator. In particular, the present invention provides a recombinant Aspergillus oryzae strain capable of expressing chitin deacetylase obtained from Mucor rouxii. The present invention also relates to a method for producing chitin deacetylase by a recombinant fungal strain. In a second aspect, the present invention relates to a recombinant yeast strain, capable of expressing chitin deacetylase, which includes an expression vector that contains a nucleic acid molecule encoding chitin deacetylase, a suitable promoter and a transcription terminator. In particular, the present invention provides a recombinant Pichia pastoris strain capable of expressing chitin deacetylase obtained from Mucor rouxii. The present invention also relates to a method for producing chitin deacetylase by a recombinant yeast strain. The present invention further relates to purified recombinant chitin deacetylase enzyme.

### RELATED APPLICATIONS

This application claims the priority of U.S. Application No. 60/447,047, filed Feb. 12, 2003 which is incorporated herein by reference in its entirety.

### FIELD OF THE INVENTION

The present invention relates to the field of recombinant DNA technology.

In a first aspect, the present invention relates to a recombinant fungal strain capable of expressing and secreting chitin deacetylase. The present invention further relates to a method for producing chitin deacetylase by a recombinant fungal strain and the purified recombinant chitin deacetylase enzyme obtained by said method.

In a second aspect, the present invention relates to a recombinant yeast strain capable of expressing chitin deacetylase. The invention also relates to a method for producing chitin deacetylase by a recombinant yeast strain and the purified recombinant chitin deacetylase enzyme obtained by said method.

### BACKGROUND OF THE INVENTION

After cellulose, chitin is the world's most abundant, easily obtained, and renewable biological material. It is a natural product synthesized by a wide variety of organisms. Several billion tons of the material are produced annually. Chitin is a carbohydrate polymer, the N-acetylated polymer of beta-(1-4)-linked N-acetylglucosamine, or poly-N-acetyl glucosamine. Chitin is a cell wall constituent replacing cellulose or sometimes occurring together with cellulose. In animals, chitin is usually organized as a cuticle at one surface of the epithelial tissue. Although structurally similar to cellulose, chitin has distinctly different chemical properties. It is an extremely insoluble material, with limited industrial applicability.

The deacetylated derivative of chitin, chitosan, is a much more tractable material with a broad and impressive array of practical applications. Chitosan is positively charged, thus, it can be used as a protein precipitant and a metal chelating agent. It can be formulated as a solution, gel, membrane, film or fiber. Such formulations are useful, for example, in the areas of precious metal recovery, crop protection, chromatography, and enzyme immobilization. Chitosan is a biologically safe, non-immunogenic, material making it ideal for use in the agricultural, food, drug and cosmetic industries. It can form complexes with other natural polymers, such as collagen and keratin, to form materials with unique biomedical properties. For example, such materials can be used as wound healing accelerants, components of artificial skin and blood vessels, anticoagulants, and controlled drug release vehicles.

The enzyme chitin deacetylase (CDA) [EC 3.5.1.41] catalyzes the conversion of chitin to chitosan by deacetylation of N-acetylglucosamine residues. This enzyme is suitable for use in a process for preparing chitosan. Chitin deacetylase activity was first identified in extracts of the fungus Mucor rouxii (Araki et al. Eur. J. Biochem. 1975, 55:71). The enzyme chitin deacetylase is produced by a variety of genera including, for example, Mucor, Phycomyces, Absidia, and Choanephora. Other potentially useful genera include Zygorhynchus, Actinomucor, Circinella, Rhizopus, Colletotrichum and Rhizomucor. Chitin deacetylase has been recently purified to homogeneity from M. rouxii (Kafetzopoulos et al. PNAS USA 1993, 90:2564) and Colletotrichum lindemuthianum (Tokuyasu et al. Chitin Enzymology vol 2 Atec Edizioni, Italy, 1996, 397) and further characterized. U.S. Pat. No. 6,004,795 discloses the isolation and sequencing of a cDNA of Mucor rouxii encoding chitin deacetylase.

The development of a process of chitosan production by an enzymatic way needs high levels of active chitin deacetylase. Some microorganisms are natural producers of chitin deacetylase but they produce low levels of the enzyme. Moreover the process for enzyme isolation is long and time-consuming. Such a process is expensive and of low economical benefits. It is not compatible with a production of chitin deacetylase on an industrial scale and with industrial applications of the enzyme.

In addition, if crude deacetylase extracts isolated from fungal mycelium are used in the preparation of chitosans, no complete deacetylation of the chitosans can be obtained. The use of a crude enzyme preparation containing chitosanolytic and/or chitinolytic enzymes, leads to partial hydrolysis of chitin and chitosan into short fragments polysaccharide. Even a pre-purification of the crude enzyme extract by acidification in order to inhibit the contaminant hydrolytic activity in the crude extract, does not avoid the presence of residual amounts of hydrolytic enzymes in the extract which can degrade the polysaccharide chains (Kolodziejska et al. Bulletin Sea Fisheries Institute, 2000, 150:2). Moreover, the use of crude preparations containing both enzymes leads to a decrease of the yield of deacetylation probably due to a competition for the substrate between chitin deacetylase and chitinase.

Heterologous expression of CDA, in particular chitin deacetylase genes from C. lindemuthianum (Tokuyasu et al. 1999. J. Biosci. Bioeng., 87 (4), 418-423) and from S. cerevisiae (Martinou et al. Protein Expression and Purification, 24, 111-116) has been reported in E. coli. However, this technique has important disadvantages. Expression of chitin deacetylase in prokaryotic systems resulted in the lack of glycosylation. It has been previously reported that deglycosylation of chitin deacetylase from several microorganisms resulted in complete loss of enzyme activity which could not be easily restored. For that reason, the use of prokaryotic systems to express chitin deacetylase originating from eukaryotic organisms is not adequate. In addition, processes based on the use of prokaryotic microorganisms expressing chitin deacetylase for producing chitosan from chitin are therefore not useful.

It is therefore an object of the present invention to provide an improved method for preparing high amounts of chitin deacetylase, suitable for use in a process for preparing chitosan from chitin. In particular, it is an object to prepare high levels of chitin deacetylase in recombinant microorganisms. It is yet another object of the present invention to provide high levels of purified chitin deacetylase, which are particularly suitable for use in a process of preparing chitosan from chitin.

### SUMMARY

According to a first aspect of the invention, there is provided a recombinant fungal strain as specified in claims 1-9 and its use in a method as specified in claims 10-19, and purified recombinant chitin deacetylase obtainable by said method, as specified in claim 20. The invention relates to a recombinant fungal strain, and preferably a recombinant filamentous fungal strain, capable of expressing heterologous chitin deacetylase under the control of a suitable promoter. Said promoter can be a constitutive or regulated promoter. The strain preferably is a filamentous fungus, and more preferred a species belonging to the genera Aspergillus, Emericella, Trichoderma, Achlya, Neurospora, Phanerochaete, Tolyplocadium or Penicillium. The present invention further provides in another embodiment, an improved method for the production of chitin deacetylase enzyme (CDA). The present invention provides a method which enables to obtain high levels of chitin deacetylase by recombinant DNA technology. More in particular, the present invention is related to a method of producing heterologous chitin deacetylase by cultivating a recombinant fungal strain capable of expressing chitin deacetylase under the control of a suitable constitutive or inducible promoter. In this method, the enzyme is produced by recombinant DNA techniques in which an isolated DNA sequence encoding a chitin deacetylase is expressed from a DNA expression construct in said fungal strain. The invention relates to a unique and effective process for preparing high levels of recombinant chitin deacetylase enzyme in a fungal strain, that is essentially free of any trace activity of hydrolytic enzymes, such as chitin or chitosan degrading enzymes or the like, that may lead to degradation of the polysaccharide chains of chitin or chitosan into short fragments polysaccharide. The term “essentially free” as used in the present invention refers to a preparation of CDA enzyme that has no significant chitin or chitosan degrading activity. Methods for measuring chitin or chitosan degrading activity are well-known in the art and are explained into more detail below. The invention also relates to preparations of recombinant chitin deacetylase, which are essentially free of any trace activity of chitin or chitosan degrading enzymes, such as chitinases or chitosanases or the like. Purified chitin deacetylase obtained by a method according to the present invention is particularly suitable for being used in a method for converting chitin to chitosan by contacting the chitin with suitable amounts of the recombinant CDA enzyme. The enzymatic conversion of chitin to chitosan provides an attractive alternative to presently used methods, which suffer from a variety of technical drawbacks. In particular, since according to the present method chitin deacetylase enzymes preparations are obtained, which are essentially free of any trace activity of chitin or chitosan degrading enzymes, the use of such chitin deacetylase in a process of preparing chitosan from chitin enables to avoid unwanted degradation and hydrolysis of chitin or chitosan polymers.

According to a second aspect of the invention, there is provided a recombinant yeast strain as specified in claims 21-35 and its use in a method as specified in claims 36-41, and purified recombinant chitin deacetylase obtainable by said method, as specified in claim 42. The invention is related to a recombinant yeast strain capable of expressing chitin deacetylase under the control of a suitable promoter. In present invention also provides a method of producing heterologous chitin deacetylase (CDA) by fermentation of a recombinant yeast strain, preferably a Pichia yeast. In this method, the enzyme is produced by recombinant DNA techniques in which an isolated DNA sequence encoding a chitin deacetylase is expressed from a DNA expression construct in said yeast strain. The present invention relates to a unique and effective process for preparing high levels of chitin deacetylase enzyme, which is essentially free of any trace activity of chitin or chitosan degrading enzymes, such as chitinases or chitosanases or the like. The invention thus provides a method which enables to obtain chitin deacetylase enzyme preparations, with essentially no residual amounts of hydrolytic enzymes. The invention also relates to preparations of recombinant chitin deacetylase, which are essentially free of any trace activity of chitin or chitosan degrading enzymes, such as chitinases or chitosanases or the like. Purified chitin deacetylase obtained by a method according to the present invention is particularly suitable for being used in a method for converting chitin to chitosan by contacting the chitin with suitable amounts of the recombinant CDA enzyme. The enzymatic conversion of chitin to chitosan provides an attractive alternative to presently used methods, which suffer from a variety of technical drawbacks. In particular, since according to the present method chitin deacetylase enzymes preparations are obtained, which are essentially free of any trace activity of chitin or chitosan degrading enzymes, the use of such chitin deacetylase in a process of preparing chitosan from chitin enables to avoid unwanted degradation and hydrolysis of chitin or chitosan polymers.

### DETAILED DESCRIPTION OF THE INVENTION

Recombinant Fungal Strain and its Use in a Method for Preparing Chitin Deacetylase

In a first aspect, the invention relates to a recombinant fungal strain, and preferably a recombinant filamentous fungal strain, capable of expressing heterologous chitin deacetylase under the control of a suitable promoter. The term “recombinant fungal strain” or “recombinant fungus” are used herein as synonyms and both refer to a fungus that contains a heterologous nucleic acid molecule, such as a cloning vector or expression vector. The term “heterologous” refers to a DNA molecule, or a population of DNA molecules, that does not exist naturally within a given host cell. An “expression vector” is defined as a nucleic acid molecule containing a gene, usually a heterologous gene, that is expressed in a host cell. Typically, an expression vector comprises a transcription promoter, a protein coding sequence consisting preferably in a cDNA, and a transcription terminator. Gene expression is always placed under the control of a promoter, and such a gene is said to be “operably linked to” the promoter. The term “operative linkage” refers to the positioning of the promoter relative to the gene product such that transcription of the gene is regulated by the promoter. Such positioning is well known in the art and involves positioning the promoter upstream (5′) of the gene so that no transcription termination signals are present between the promoter and the gene. Hereafter, construction of a recombinant fungal strain by DNA techniques will be explained into more detail.

In an embodiment, the invention relates to a method wherein a recombinant fungal strain is constructed comprising an expression vector that contains a promoter, a nucleic acid molecule encoding chitin deacetylase, a selectable marker sequence, a secretion signal sequence, and a transcription terminator. The different elements are fused so as to constitute a functional chimeric gene.

In a preferred embodiment the recombinant fungal strain is a filamentous fungus. The term “filamentous fungus” refers to a saprophytic microorganism that can be cultured alone in vitro as free mycelia. The microorganism forms multinucleated, tubular filaments called hyphae that are functionally coencytial and grows by apical extension. Filamentous fungi have proved to be extremely useful in industry to produce metabolites such as peptides, enzymes, organic acids and antibiotics. More recently fungi have been developed as host organisms for the production of heterologous recombinant proteins. Filamentous fungi are very attractive as they have many advantages compared with other expression systems. These advantages include: the ability to produce and secrete very large amounts of proteins; the expression of proteins in the correctly folded and functional form (in particular including post-translational modifications such as glycosylation and disulfide bond formation) which is impossible with bacterial systems; several filamentous fungi have the GRAS status, which allows food-grade applications and facilitate biomedical applications; stable recombinants can be isolated, thus enabling controlled strain breeding. In a preferred embodiment, the filamentous fungus is selected from the genus Aspergillus, Emericella, Trichoderma Achlya, Neurospora, Phanerochaete, Tolyplocadium or Penicillium. In an example the fungus is from the genus Aspergillus. In another example, the filamentous fungus is Aspergillus oryzae, Aspergillus niger, Emericella nidulans (=Aspergillus nidulans), Penicillium chrysogenum, Trichoderma reesei or Trichoderma viride. In a preferred embodiment, the fungal strain is Aspergillus oryzae strain MUCL 14492 or strain MUCL 31310. In another preferred embodiment, the fungal strain is Aspergillus niger strain MUCL 3766, MUCL 19002 or MUCL 13608. In another preferred embodiment, the fungal strain is Emericella nidulans strain MUCL 3563. In yet another preferred embodiment, the fungal strain is Penicillium chrysogenum strain MUCL 28658 or MUCL 29142.

In another embodiment, the invention relates to a recombinant fungal strain comprising an expression vector. The vectors used for chitin deacetylase expression in the present invention are preferably vectors allowing integration in the filamentous fungal genome by homologous or random recombination. In a preferred example, the vector is a vector from the pUT family vectors for integration in an Aspergillus strain. The Aspergillus expression system is a very interesting system for producing high levels of functionally recombinant proteins. The system combines advantages like high-level expression, high-level secretion, inexpensive growth of the fungi, and the advantages of an eukaryotic system like protein processing, folding and post-translational modifications. Examples of suitable vectors include but are not limited to pUT765, pUT970, pUT971, which are commercially available at Cayla (Toulouse, France). Other suitable vectors include but are not limited to pNAN (Ozeki K. et al., Biosciences Biotechnology Biochemistry, 1996, 60, 383-389); pAN56 (Mullaney E. J. et al., Mol. Gen. Genet., 1985, 199, 37-45), pAN56-2H (available in EMBUGenBank: accession number NCBI gi: 474930) or pAB 520 (Austin B. et al., Gene, 1990, 93, 197-162).

In another embodiment, the invention relates to a method wherein a recombinant fungal strain is constructed comprising an expression vector containing a constitutive or a regulated promoter, driving expression of the CDA gene in the recombinant fungus. The term “promoter” refers to a nucleotide sequence at the 5′end of a structural gene which directs the initiation of transcription. The term “constitutive promoter” refers to a promoter of a gene that shows an essentially continuous and constant level of transcription. Suitable constitutive promoters for use in the present invention include but are not limited to promoters selected from the group comprising promoter sequences of A. nidulans triose-phosphate-isomerase dehydrogenase (tpi), (Upshall et al. 1987. Bio/Technology, 5, 1301-1304) A. nidulans glyceraldehyde-3-phosphate dehydrogenase (gpdA) (Upshall et al. 1987; Punt et al., 1991. J. Biotechnol., 17, 19-34; Archer et al., 1990. Biotechnology, 8, 741-745; Kolar et al., 1988. gene, 62, 127-134) and A. nidulans ATP-synthase sub-unit 9 (oliC) genes (Upshall et al. 1987. Bio/Technology, 5, 1301-1304; Van Gorcom et al. 1986. Gene, 48, 211-217; Knowles et al. 1987. Antonie van Leeuwenhoek, 53, 335-341), of A. niger alcohol dehydrogenase (adhA) gene (Upshall et al. 1987; Saunders et al. 1989. Trends Biotechnol., 7, 283-287) or of A. oryzae α-amylase (amy) gene (Boel et al. 1987; EP 238,023).

The term “regulated” or “induced” promoter refers to a promoter of a gene that regulates the level of transcription in response to an inducing agent. The term “inducing agent” refers to a particular condition, such as but not limited specific substrates, stress conditions, specific temperature or pH conditions, etc, which initiates the transcription of the gene encoding the interest protein. The term “promoter induction” refers to conditions, which increase the transcription level of the gene of interest, and in particular herein the expression of the CDA gene. Suitable inducible promoters for use in the present invention can for example be but are not limited to promoter sequences of A. niger glucoamylase (glaA) gene or A. awamori glucoamylase (glaA) gene (Gwinne et al. 1987. Bio/Technology, 5, 713-719 Cullen et al., 1987. Bio/Technology, 5, 369-376), of A. nidulans alcohol dehydrogenase (alcA, alcC) (Gwinne et al. 1987) or of A. nidulans acetamidase (amdS) genes (Upshall et al. 1987. Bio/Technology, 5, 1301-1304; Turnbull et al. 1989. Bio/Technology, 7, 169-173), or of Trichoderma viride cellobiohydrolase gene (Knowles et al. 1987. Antonie van Leeuwenhoek, 53, 335-341) or of Trichoderma reesei cellobiohydrolase I (cbh1) gene (Nyyssbnen & Keranen, 1995. Current Genetics, 28, 71-79). In a preferred embodiment, a promoter used in the present invention includes the gpdA promoter from Aspergillus nidulans, which is part of a gene encoding glyceraldehyde-3-phosphate-dehydrogenase. This promoter is functional in several genera and species, like for example Penicillium chrysogenum (Kolar et al. 1988. Gene, 62, 127-134), Aspergillus nidulans (Punt P et al. 1991. Journal of Biotechnology, 17, 19-34) or Aspergillus niger (Archer et al. 1990. Bio/Technology, 8, 741-745).

A cDNA encoding chitin deacetylase is used to construct the recombinant fungal strain for chitin deacetylase expression. In a preferred embodiment said cDNA encoding chitin deacetylase is obtained from Mucor rouxii. The cDNA sequence of Mucor rouxii encoding chitin deacetylase used in the present invention is disclosed in U.S. Pat. No. 6,004,795 (Thireos & Kafetzopoulos). It is noted that the DNA sequence used in the present invention is the fragment corresponding to the chitin deacetylase coding region (corresponding to the mature protein). The CDA sequence used in the present invention differs from the sequence disclosed in U.S. Pat. No. 6,004,795 in that for cloning purposes additional sequences were added to the CDA sequence at the 3′ and 5′ end of the gene sequence.

In yet another embodiment, the present method comprises construction of a recombinant fungal strain comprising an expression vector that comprises a selectable marker gene. The term “auxotrophic marker” refers to a marker that is used to complement specific nutritional requirements in mutant strains which are auxotrophic for the nutrient in question due to the absence of a functional chromosomal copy of the marker gene. The term “dominant marker gene” refers to a marker that allows selection on the basis of resistance to antibiotics or to toxic compounds, avoiding the requirement of creating a mutant auxotrophic strain. In an example said selectable marker is an auxotrophic marker gene including but not limited to the trpC gene in A. nidulans (Yelton et al., 1984. PNAS USA, 81, 1470-1474), the argB gene in A. nidulans (John & Peberdy, 1984. Enzymes Microbiol. Technol., 6, 386-389), the pyr4 from Neurospora crassa (Ballance et al., 1983. Biochem. Biophys. Res. Commun., 112, 284-289). In another example said selectable marker is a dominant marker gene including but not limited to the marker genes that confer oligomycin resistance such as but not limited to the Tn5 neomycin phosphotransferase gene from E. coli (Rambosek & Leach, 1987. Rev. Biotechnol., 6, 357-373) the hygromycin B phosphotransferase gene from E. coli (Punt at al., 1987. Gene, 56, 117-124) or the Aspergillus niger oliC3 gene (Ward et al., 1988. Curr. Genet. 14, 37-42). In another example the selectable marker gene is an antibiotic resistance gene such as, but not limited to the phleomycin resistance gene (Sh ble gene) originating from Streptoalloteichus hindustanus, or the AmdS gene originating from Aspergillus nidulans that allows the cells to grow on acetamide as sole nitrogen source (Hynes et al, Mol. Cell. Biol., 3, pp 1430-1439, 1983). The selectable marker allows the transformed cells to grow under conditions in which untransformed cells cannot multiply. In a preferred example, the used vectors in the present invention carry a dominant selection marker gene such as the Sh ble gene, or the AmdS gene. In this last case, the selectable marker can be located on a separate vector in which case cells are co-transformed with the said separate vector such as the p3SR2 plasmid carrying the AmdS gene.

The invention relates to a method wherein a recombinant fungal strain is constructed comprising an expression vector that carries a secretion signal sequence. The term “secretion signal sequence” refers to a nucleotide sequence coding for a peptide at the N-terminus of the primary translation product that is responsible for directing secretory proteins into the secretion pathway. Examples of suitable secretion signal sequences for use in the present invention comprise but are not limited to secretion signal sequences from higher eukaryotes such as those mentioned in Salovuori et al. (1987. Bio/technology, 5, 152-156), mammalian signal sequences including those for chymosin (Cullen et al., 1987. Bio/technology, 5, 369-376) and interferon (Gwynne et al., 1987. Bio/technology, 5, 713-719), signal sequence of hen egg white lysozyme (Archer et al. 1990. Bio/Technology, 8, 741-745). The CDA sequence can also be fused with the complete coding region from a strongly expressed homologous protein gene like for example fungal glucoamylase (Ward et al., 1990. Bio/Technology, 8, 435-438; Contreras et al., 1991. Biotechnology, 9, 378-381) or with truncated forms of A. niger glucoamylase sequence (Jeenes et al., 1993. FEMS Microbiology Lettres, 107, 267-272) or T. reesei cellobiohydrolase 1, (CBHI) (Nyyssonen et al. 1993. Bio/Technology, 11, 591-595). In a particularly preferred embodiment, the secretion signal sequence for use in the present invention consists of the Ssa sequence which is a synthetic DNA coding for the cellobiohydrolase I signal sequence of Trichoderma reesei (Calmels T. P. G. et al., J. Biotechnol., 1991, 17, 51-66.)

In a further embodiment, the present invention relates to the expression of chitin deacetylase as a translational fusion to the C-terminus of another protein in a filamentous fungus. In particular, the nucleic acid molecule encoding chitin deacetylase is expressed as a translational fusion to the C-terminus of the selection marker protein such that chitin deacetylase is expressed as a C-terminal fusion protein. The recombinant fungal strain comprises an expression vector wherein the nucleic acid molecule encoding chitin deacetylase is expressed in frame translational fusion downstream of the selection marker coding sequence such that chitin deacetylase is expressed as a C-terminal fusion protein. The fused protein, which is secreted, serves as a carrier to improve the expression and the secretion of the CDA protein. Preferably, in the used vectors, the chitin deacetylase sequence is fused in frame to the phleomycin resistance gene (Sh ble gene) from Streptoalloteichus hindustanus, and preceded by a secretion signal sequence (e.g. Ssa see above). The fusion protein is efficiently secreted and the release of active chitin deacetylase is obtained by in vivo or in vitro proteolytic cleavage in the junction peptide between Sh ble and chitin deacetylase protein. Therefore, a recognition sequence for a KEX2-like endoproteolytic cleavage site, similar to that found in many fungal precursor proteins (Calmels et al., Journal of Biotechnology, 17, pp 51-66, 1991), or a recognition sequence for a Factor X cleavage site (Himmelspach M. et al., Thrombosis Research, 2000, 97, 51-67) is preferably inserted between the two proteins (Sh ble and CDA) to allow the cleavage of the fusion protein.

In another embodiment, the present method comprises construction of a recombinant fungal strain comprising an expression vector that comprises additional nucleotide sequences provided at the 5′ end and/or at the 3′ end of the nucleic acid molecule encoding chitin deacetylase. Said additional sequences, so-called ‘tag sequences’, can be provided at the 5′ and 3′ terminal end of the chitin deacetylase cDNA sequence, such that additional amino acids are provided at the N- or the C-terminal site respectively of the expressed enzyme. Such tag sequences may be advantageously applied in the purification process of the expressed enzyme.

In a particularly preferred example, the recombinant fungal strain according to the present invention comprises a recombinant Aspergillus oryzae strain capable of expressing chitin deacetylase obtained from Mucor rouxii, wherein said recombinant fungus is provided with a pUT expression vector that comprises the cDNA sequence encoding chitin deacetylase from Mucor rouxii, the gpdA promoter sequence from A. nidulans, the Sh ble selectable marker sequence from Streptoalloteichus hindustanus, the Ssa secretion signal sequence from Trichoderma reesei and optionally additional (tag) sequences provided at the 5′ and/or 3′ terminal end of nucleic acid molecule encoding CDA. In another example, the present invention provides a recombinant fungal strain which corresponds to a strain represented in Table 1.

TABLE 1
Non-limiting examples of recombinant fungal strains according to the
present invention

| | | | Secretion signal | | Selectable | N-terminal TAG | C-terminal TAG |
|---|---|---|---|---|---|---|---|
| Strain | vector | promoter | sequence | CDA | marker gene | sequence | sequence |
| A. oryzae | pUT970 | gpdA | Ssa | M. rouxii | Sh ble | — | — |
| A. oryzae | pUT970 | gpdA | Ssa | M. rouxii | Sh ble | 6XHIS | — |
| A. oryzae | pUT970 | gpdA | Ssa | M. rouxii | Sh ble | — | 6XHIS |
| A. oryzae | pUT 765 | gpdA | Ssa | M. rouxii | Sh ble | — | — |
| A. oryzae | pUT 765 | gpdA | Ssa | M. rouxii | Sh ble | 6XHIS | — |
| A. oryzae | pUT 765 | gpdA | Ssa | M. rouxii | Sh ble | — | 6XHIS |
| A. oryzae | pUT 971 | gpdA | Ssa | M. rouxii | Sh ble | — | — |
| A. oryzae | pUT 971 | gpdA | Ssa | M. rouxii | Sh ble | 6XHIS | — |
| A. oryzae | pUT 971 | gpdA | Ssa | M. rouxii | Sh ble | — | 6XHIS |
| A. niger | pUT970 | gpdA | Ssa | M. rouxii | Sh ble | — | — |
| A. niger | pUT970 | gpdA | Ssa | M. rouxii | Sh ble | 6XHIS | — |
| A. niger | pUT970 | gpdA | Ssa | M. rouxii | Sh ble | — | 6XHIS |
| A. niger | pUT765 | gpdA | Ssa | M. rouxii | Sh ble | — | — |
| A. niger | pUT765 | gpdA | Ssa | M. rouxii | Sh ble | 6XHIS | — |
| A. niger | pUT765 | gpdA | Ssa | M. rouxii | Sh ble | — | 6XHIS |
| A. niger | pUT971 | gpdA | Ssa | M. rouxii | Sh ble | — | — |
| A. niger | pUT971 | gpdA | Ssa | M. rouxii | Sh ble | 6XHIS | — |
| A. niger | pUT971 | gpdA | Ssa | M. rouxii | Sh ble | — | 6XHIS |
| E. nidulans | pUT970 | gpdA | Ssa | M. rouxii | Sh ble | — | — |
| E. nidulans | pUT970 | gpdA | Ssa | M. rouxii | Sh ble | 6XHIS | — |
| E. nidulans | pUT970 | gpdA | Ssa | M. rouxii | Sh ble | — | 6XHIS |
| E. nidulans | pUT765 | gpdA | Ssa | M. rouxii | Sh ble | — | — |
| E. nidulans | pUT765 | gpdA | Ssa | M. rouxii | Sh ble | 6XHIS | — |
| E. nidulans | pUT765 | gpdA | Ssa | M. rouxii | Sh ble | — | 6XHIS |
| E. nidulans | pUT971 | gpdA | Ssa | M. rouxii | Sh ble | — | — |
| E. nidulans | pUT971 | gpdA | Ssa | M. rouxii | Sh ble | 6XHIS | — |
| E. nidulans | pUT971 | gpdA | Ssa | M. rouxii | Sh ble | — | 6XHIS |
| P. chrysogenum | pUT970 | gpdA | Ssa | M. rouxii | Sh ble | — | — |
| P. chrysogenum | pUT970 | gpdA | Ssa | M. rouxii | Sh ble | 6XHIS | — |
| P. chrysogenum | pUT970 | gpdA | Ssa | M. rouxii | Sh ble | — | 6XHIS |
| P. chrysogenum | pUT765 | gpdA | Ssa | M. rouxii | Sh ble | — | — |
| P. chrysogenum | pUT765 | gpdA | Ssa | M. rouxii | Sh ble | 6XHIS | — |
| P. chrysogenum | pUT765 | gpdA | Ssa | M. rouxii | Sh ble | — | 6XHIS |
| P. chrysogenum | pUT971 | gpdA | Ssa | M. rouxii | Sh ble | — | — |
| P. chrysogenum | pUT971 | gpdA | Ssa | M. rouxii | Sh ble | 6XHIS | — |
| P. chrysogenum | pUT971 | gpdA | Ssa | M. rouxii | Sh ble | — | 6XHIS |
| T. viride | pUT970 | gpdA | Ssa | M. rouxii | Sh ble | — | — |
| T. viride | pUT970 | gpdA | Ssa | pUT970 | Sh ble | 6XHIS | — |
| T. viride | pUT970 | gpdA | Ssa | pUT970 | Sh ble | — | 6XHIS |
| T. viride | pUT765 | gpdA | Ssa | pUT970 | Sh ble | — | — |
| T. viride | pUT765 | gpdA | Ssa | pUT970 | Sh ble | 6XHIS | — |
| T. viride | pUT765 | gpdA | Ssa | pUT970 | Sh ble | — | 6XHIS |
| T. viride | pUT971 | gpdA | Ssa | pUT970 | Sh ble | — | — |
| T. viride | pUT971 | gpdA | Ssa | pUT970 | Sh ble | 6XHIS | — |
| T. viride | pUT971 | gpdA | Ssa | pUT970 | Sh ble | — | 6XHIS |
| T. reesei | pUT970 | gpdA | Ssa | M. rouxii | Sh ble | — | — |
| T. reesei | pUT970 | gpdA | Ssa | M. rouxii | Sh ble | 6XHIS | — |
| T. reesei | pUT970 | gpdA | Ssa | M. rouxii | Sh ble | — | 6XHIS |
| T. reesei | PUT765 | gpdA | Ssa | M. rouxii | Sh ble | — | — |
| T. reesei | PUT765 | gpdA | Ssa | M. rouxii | Sh ble | 6XHIS | — |
| T. reesei | PUT765 | gpdA | Ssa | M. rouxii | Sh ble | — | 6XHIS |
| T. reesei | pUT971 | gpdA | Ssa | M. rouxii | Sh ble | — | — |
| T. reesei | pUT971 | gpdA | Ssa | M. rouxii | Sh ble | 6XHIS | — |
| T. reesei | pUT971 | gpdA | Ssa | M. rouxii | Sh ble | — | 6XHIS |

In another particularly preferred embodiment, the invention provides a recombinant fungal strain having accession number IHEM 20351. A recombinant fungal strain obtained according to the present invention has been deposited to the BCCM-IHEM, biomedical fungi and yeasts collection, Scientific Institute of Public Health, Louis Pasteur, Brussels Belgium on Feb. 10, 2004. The deposited strain has the characteristics of the allocated accession number IHEM 20351.

In another aspect, the invention relates to a method for producing high levels of chitin deacetylase using a recombinant fungal strain. The present invention provides a method for culturing a recombinant fungal strain, expressing a chitin deacetylase gene by said strain and secreting high levels of the CDA protein. More particularly, the method comprises the steps of:

constructing a recombinant fungal strain capable of expressing chitin deacetylase by recombinant DNA techniques,

preparing a culture comprising spores of said recombinant fungal strain,

inoculating a suitable amount of spores of said recombinant fungal strain in a suitable medium and incubating said recombinant fungal strain in said medium for a suitable period of time,

feeding for a suitable period of time said incubated recombinant fungal strain with a suitable substrate which controls proliferation of the fungal strain,

clarifying the medium such that fungal mycelium is removed from the medium and the supernatant of the medium which comprises chitin deacetylase is retained, and

isolating said chitin deacetylase from said supernatant by means of chromatographic techniques.

The first step in the present method relates to the construction of a recombinant fungal strain capable of expressing chitin deacetylase. Preferably, a recombinant strain as described above is constructed.

Practically, the chitin deacetylase sequence is amplified by PCR technique with a M. rouxii cDNA sequence as template and primers designed on the basis of the sequence of the fragment and containing appropriate restriction sites for the cloning into the vector. The obtained PCR products are cloned in the vectors that were previously linearized by restriction with appropriate enzymes depending of the used vector. Appropriate restriction sites are provided for cloning in the fungal expression vectors, which preferably comprise AsuII-BamHI for cloning in pUT970, pUT765 and pUT971 vectors. Final constructed vectors are used to transform fungal strains. Transformation can be performed according to methods known to a person skilled in the art. Preferably, transformation is performed by a method comprising the preparation of protoplasts and use of polyethylene glycol (PEG) (Tilburn et al. 1983. Gene, 26, 205-221) or the preparation of protoplasts and electroporation of the said protoplasts (Fowler 1990. Curr. Genet., 18, 537-545). Another suitable method of transformation consists in the preparation of conidia and electroporation of said conidia (Sanchez & Aguirre, 1996. Fungal genetics Newsletters, 43, 48-51; Weidner et al., 1998. Current Genetic, 33, 378-385).

According to the characteristics of the used strain, the conditions of transformation and selection may be different. If the strain is sensitive to phleomycin, the strain can be transformed with only a pUT vector and transformed cells are selected on the basis of phleomycin resistance. If for instance the strain is naturally resistant to phleomycin, the strain is co-transformed with a second vector, e.g. carrying the AmdS gene, and the transformed cells are selected on medium containing acetamide as sole nitrogen source. Before screening for enzyme expression, transformants are preferably analyzed by PCR using specific primers in order to determine if the gene of interest has integrated into the fungal genome. Subsequently, the transformants are screened for their ability to express chitin deacetylase when they are cultivated in liquid medium. Chitin deacetylase activity can be measured in the culture supernatant by well-known methods (Araki et al. 1975. Eur. J. Biochem., 55, 71-78).

In a further embodiment, the method comprises the step of inoculating an amount of spores of said recombinant fungal strain, preferably comprised between 0.5×10⁵ and 1.0×10⁸ spores per ml medium in a suitable culture medium. Spores are obtained from a fresh culture on agar plate or from a frozen glycerol stock. For long-term storage, recombinant fungal spores are preferably kept at −70 C° in a solution containing 100 g l⁻¹ powder skimmed milk and 100 ml l⁻¹ glycerol.

According to a subsequent step of the present method the recombinant fungal strain is cultivated in a liquid medium comprising adequate carbon sources, nitrogen and nutrients. As with most expression systems, the behaviours of particular constructs in a culture can vary and the same protocol cannot be used with all of them. Moreover, gene expression depends on careful control of culture conditions. To obtain an optimal control of cell growth and protein expression the choice of suitable parameters therefore is crucial. A preferred culture medium is AMM or modified AMM medium, which composition is described hereafter. AMM suitably comprises: 5 g l⁻¹ yeast extract; 6 g l⁻¹ NaNO₃; 10-30 g l⁻¹ D-glucose; 26 g l⁻¹ KCl; 76 g l⁻¹ KH₂PO₄; 26 g l⁻¹ MgSO₄; trace elements solution 1 ml l⁻¹ and is prepared with deionized water. In modified AMM medium, D-glucose is replaced by sucrose, starch, mixture of sugars obtained after partial starch hydrolysis (fluitex, glucidex), malt extract, maltodextrin, or a combination of those elements. Another possible composition of culture medium is: 20 g l⁻¹ D-glucose; 10 g l⁻¹ peptone; 1 g l⁻¹ yeast extract; 5 g l⁻¹ (NH₄)₂SO₄; 1 g l⁻¹ K₂HPO₄; 1 l⁻¹ NaCl; 5 g l⁻¹ MgSO₄.7H₂O; 0.1 g l⁻¹ CaCl₂. Trace elements solution comprises: 76 μM ZnSO₄; 178 μM H₃BO₃; 25 μM MnCl₂; 18 μM FeSO₄; 7.1 μM CoCl₂; 6.4 μM CuSO₄; 6.2 μM Na₂MoO₄; 174 μM EDTA. AMM medium can also be supplemented with acid hydrolyzed casein (e.g. casamino acids). It will be clear to the person of skill in the art that the composition of the culture medium may vary. For example, sucrose, starch, partially hydrolyzed starch (fluitex, glucidex), or other carbon sources, alone or in combination with each other can be used instead of D-glucose.

The pH of said culture medium is adjusted before sterilization of the medium. In a preferred embodiment the pH of said medium is adjusted to a pH of 4.5 to 7.0, and for instance to a pH of 5 to 5.5, before inoculation of said spores of the recombinant fungal strain in the medium. The pH of the culture medium is preferably adjusted with a solution of sodium hydroxide or with a solution of nitric acid. However, it is clear that the pH of said medium could also be adjusted with other solutions.

In another embodiment, the present method further comprises the step of supplementing the medium, after the pH of the culture medium has been adjusted, with a solution comprising metal trace elements such as e.g., iron, zinc, copper, magnesium, manganese, calcium, cobalt, preferably in amount ranging from 0.5 to 2.0 ml l⁻¹ and more preferably in an amount of 1.0 ml l⁻¹ and/or vitamins such as biotin, nicotinic acid, thiamine, nicotinamide preferably in amount ranging from 0.1 to 2 mg per liter medium. This solution is previously sterilized, e.g. by filtration on a 0.22 μm pore size filter. It will be clear that the solution and the amount of its particular ingredients may vary.

The inoculated culture is preferably grown at a temperature comprised between 25° C. and 35° C. and for instance between 28° C. and 30° C., with low orbital shaking, for instance ranging from 100 to 250 rpm. The recombinant fungal strain is incubated in said medium for a suitable period of time, preferably comprised between 48 and 144 hours. During incubation, the culture is fed for a suitable period of time preferably comprised between 0 and 144 hours with a suitable substrate which controls proliferation of the fungus. Examples of suitable substrates include but are not limited to glucose, acid hydrolyzed casein (e.g. casamino acids), sucrose, starch, partially hydrolyzed starch (for example, fluitex, glucidex), maltose, maltodextrin, malt extract, soya extract, potato dextrose or other carbon sources, alone or in combination with each other.

In a further preferred embodiment, samples are harvested, e.g. every 24 hours during culture period, in order to analyze microbiological purity of the culture, protein concentration and enzyme activity. Since the cDNA encoding chitin deacetylase is cloned in a vector for secreted expression, the chitin deacetylase is produced in the extracellular medium. Mycelium is removed from the medium by filtration such that the culture broth which comprises chitin deacetylase is retained. Chitin deacetylase activity can be measured by well-known methods either directly in the culture supernatant or after dialysis against water or adequate buffer. Samples can also be concentrated by tangential ultrafiltration and diafiltration before analysis. To check on CDA activity during the culture, the ultrafiltration operation can be performed with sample volumes from 0.5 ml to 15 ml, on centrifugal concentrator units comprising membranes of 10.000 to 30.000 NMWC (nominal molecular weight cut-off). The final resulting concentration factor is 10-20 times. In some cases, chitin deacetylase activity can also be measured in the soluble intracellular fraction obtained after crushing the mycelium in an adequate buffer and centrifugation to separate the supernatant containing soluble proteins from the pellet of cell debris. Total protein amounts are measured by BCA method (kit BCA, Pierce) and protease activity is checked with Hide Powder Azur (Calbiochem).

The present invention relates in a further embodiment, to a method comprising the step of clarifying said medium by filtration, centrifugation and/or microfiltration such that fungal mycelium is removed from the medium. The culture broth (supernatant) which contains chitin deacetylase is retained.

The present invention further provides for the purification of chitin deacetylase from the culture supernatant. The supernatant contains both chitin deacetylase and enzymes having chitinase activity. For further use of chitin deacetylase in methods wherein chitin is converted into chitosan, it is essential to completely remove chitinolytic enzyme activity from enzymatic preparation, in order to avoid the hydrolysis of chitin and chitosan polysaccharides. The present invention provides a preparation comprising chitin deacetylase that is essentially free of any trace activity of chitin or chitosan degrading enzymes, such as chitinases or chitosanases or the like, which could induce hydrolysis of chitin or chitosan, when used in a process of preparing chitosan from chitin. The term “essentially free of” as used in the present invention refers to a preparation comprising CDA enzyme having a non-detectable level of chitin or chitosan degrading activity. Measurement of chitin or chitosan degrading activity is well-known in the art and may be done by electrophoresis under native conditions on polyacrylamide gels containing glycolchitin (=substrate of CDA), followed by incubation and coloration of zones of hydrolysis of the gel. Another method is based on viscosimetry: by incubating the enzymatic preparation with a polymer solution (e.g. chitin or chitosan) having a known viscosity, and by following the evolution in viscosity. If the enzymatic CDA preparation contains hydrolyzing enzymes, zones of hydrolysis will be seen on the polyacrylamide gels, and a decrease in viscosity will be detected.

In a preferred embodiment of the present method, the chitin deacetylase is isolated from said supernatant by chromatographic techniques in such a way that CDA which is essentially free of any trace activity of chitin or chitosan degrading enzymes is obtained Purification of the enzyme from the supernatant can be performed by conventional chromatographic procedures. In an embodiment, chitin deacetylase is isolated from the supernatant by hydrophobic interaction chromatography or ionic exchange chromatography, e.g. cation exchange chromatography. The combination of the methods and the purification scheme are dependent of the expected purity level. In an example, the chitin deacetylase is isolated from said supernatant by cation exchange chromatography, in such a way that conductivity of the buffer for eluting chitin deacetylase from a chromatography column is comprised between 3 and 9 ms/cm.

In another embodiment, chitin deacetylase is isolated from said supernatant by metal chelate affinity chromatography. This technique is used for isolating chitin deacetylase expression by a recombinant fungal strain, according to the invention, having an expression vector in which 3′ and/or 5′ tag sequences have been provided.

Chitin deacetylase activity can be measured by well-known methods either directly in the culture supernatant, or in the different protein fractions obtained during the purification process. Methods for measuring CDA activity are well known in the art. For instance, a method of enzyme assay includes the determination of acetic acid levels (Kolar, et al. 1988. Gene, 62, 127-134) released during the incubation of chitin deacetylase with chitinous substrates.

In yet another aspect, the present invention relates to purified recombinant chitin deacetylase, which is obtainable by the preparation method according to the present invention, wherein a recombinant fungal strain is applied. In a preferred embodiment, said purified recombinant chitin deacetylase has a molecular mass of ˜75 kDa. This molecular mass can be confirmed by western blot analysis. After gel electrophoresis in denaturing conditions and protein transfer on nylon membrane, a protein band at ˜75 kDa can be identified after immunoreaction with antibodies directed against synthetic peptides designed in the conserved domain deduced from amino acid sequence alignment of different chitin deacetylases.

Recombinant chitin deacetylase obtained according to the present invention is able to hydrolyze chitinous substrates such as chitohexaose, carboxymethylchitin, glycol chitin, insoluble colloidal chitin and partially deacetylated chitosans. Preferably, recombinant chitin deacetylase, obtained according to the present invention, can be used in a process of chitin or chitosan deacetylation. As an example, the recombinant enzyme can be used to extend the deacetylation of chitosan, from various origins. Industrial chitosan preparation methods consist of contacting the chitin with suitable amounts of chitin deacetylase enzyme obtained according to the present method. The enzymatic conversion of chitin to chitosan provides an attractive alternative to presently employed methods, which suffer from a variety of technical drawbacks. Preparations of recombinant chitin deacetylase obtained according to the present invention are particularly pure and essentially free of chitin or chitosan degrading activity. Therefore its application in above-cited enzymatic chitosan preparation processes allows producing highly deacetylated chitosan, with no loss of molecular weight and no loss of material, and no need for fractionation of the polymer chains. In particular, the use of such chitin deacetylase in a process of preparing chitosan from chitin enables to avoid unwanted degradation and hydrolysis of chitin or chitosan polymers.

In addition, recombinant chitin deacetylase, obtained according to the method of the present invention, may be particularly suitable for use in the preparation of industrial amounts of chitin and chitosan, since in accordance with the present invention it can be prepared in large quantities.

Recombinant Yeast Strain and its Use in a Method for Preparing Chitin Deacetylase

In a second aspect, the present invention relates to a recombinant yeast strain capable of expressing chitin deacetylase. The term “recombinant yeast” refers to a yeast that contains a heterologous nucleic acid molecule, such as a cloning vector or expression vector. The term “heterologous” refers to a DNA molecule, or a population of DNA molecules, that does not exist naturally within a given host cell. An “expression vector” is defined as a nucleic acid molecule containing a gene, usually a heterologous gene, that is expressed in a host cell. Typically, this gene comprises a protein encoding sequence consisting preferably in a cDNA gene. Gene expression is always placed under the control of a promoter, and such a gene is said to be “operably linked to” the promoter.

In a preferred embodiment, the invention provides a recombinant yeast strain comprising an expression vector that contains a nucleic acid molecule encoding chitin deacetylase of interest, a suitable promoter and a transcription terminator, wherein the promoter is operably linked with the nucleic acid molecule, and wherein the nucleic acid molecule is operably linked with the transcription terminator. In another embodiment, the present invention relates to a recombinant yeast strain capable of expressing chitin deacetylase under the control of an inducible promoter. In a preferred embodiment, a recombinant yeast strain capable of expressing the chitin deacetylase under the control of an inducible promoter is constructed by recombinant DNA techniques.

The origin of the recombinant yeast is not particularly limited, and the recombinant yeast is exemplified by a yeast, e.g., from the genus Saccharomyces or the genus Hansenula or Pichia. Said yeast preferably is a methylotrophic yeast. The term “methylotrophic yeast” refers to yeasts which are capable of growing in methanol and metabolizing methanol as carbon source. Examples of suitable methylotrophic yeast include, but are not limited to yeasts belonging to the genus Hansenula or the genus Pichia, which are capable of growing in methanol (The Biochemistry of Methylotrophs, 269, 1982). Preferred are methylotrophic yeasts belonging to the genus Pichia, such as Pichia pastoris and Pichia methanolica. Pichia pastoris strains are mutant strains (see review in Cereghino J. L. & Cregg J. M., FEMS Microbiology Review, 2000, 24, 45-66) derivatives of the wild-type strain NRRL-11430 (Northern Regional Research Laboratories, Peoria, Ill.). Pichia methanolica strains are mutant strains (Raymond C., U.S. Pat. No. 5,716,808) derivatives of the wild-type strain ATCC-CBS6515 (American Type Culture, Rockville, Md.). In a more preferred embodiment, yeasts used for chitin deacetylase expression are preferably Pichia pastoris strain GS115, KM71 KM71H, SMD1168, SMD1168H, or X33 and Pichia methanolica strain PMAD11 or PMAD16. The above-mentioned strains of P. pastoris and P. methanolica are well known to a person of skill in the art and commercially available from Invitrogen (San Diego, Calif., US) In a more preferred embodiment, Pichia pastoris is used to obtain the highest level of expression of recombinant chitin deacetylase. Even more preferred is the Pichia pastoris strain GS115.

According to a more preferred embodiment, a recombinant Pichia strain comprising an expression vector is used. The Pichia expression system is a very interesting system for producing high levels of functionally recombinant proteins. This system combines advantages like high-level expression, easy scale-up, inexpensive growth of the yeast, and the advantages of an eukaryotic system like protein processing, folding and posttranslational modifications. The vectors used for chitin deacetylase expression are preferably Pichia expression vectors, and preferably pFLD1α pPIC9, pHIL-S1, pPIC9K, pPIC6α, pPICZα-E or pPICZα-vectors in Pichia pastoris, and a pMETα vector in Pichia methanolica. These vectors (see review in Cereghino J. L. & Cregg J. M., FEMS Microbiology Review, 2000, 24, 45-66) are well known to a person of skill in the art and are commercially available from Invitrogen (San Diego, Calif., US). In a preferred embodiment, the pPIC9 vector of Pichia pastoris is used. In another preferred embodiment, the pPIC9K vector of Pichia pastoris is used. In yet another preferred embodiment the pPICZα vector of Pichia pastoris is used.

Vectors comprising an inducible promoter for controlling transcription and expression of the CDA gene may be used, such as those enumerated above. However, it is clear that vectors comprising a constitutive promoter for controlling transcription and expression of the CDA gene and providing constitutive expression of the CDA gene may be used as well, such as for example a pGAPZα vector in Pichia pastoris (Waterham H. R. et al., Gene, 1997, 186,37-44). This vector is well known to a person of skill in the art and is commercially available from Invitrogen (San Diego, Calif., US). When using this vector, it will be clear to a person of skill in the art that the method for producing chitin deacetylase by the recombinant yeast strain as explained below will not require the step of supplementing an inducer, since CDA is constitutively expressed in such yeast strains.

In a preferred embodiment, the expression vectors contain an inducible and tightly regulated promoter, more preferably an alcohol-inducible promoter and most preferably a methanol-inducible promoter. Illustrative methanol-inducible promoters include a Pichia pastoris alcohol oxidase 1 (AOX1) promoter, a Pichia pastoris FLD1 promoter (for methanol- or methylamine-induced expression of the gene of interest), a Pichia pastoris PEX8 promoter (PEX8 gene encodes a peroxisomal matrix protein), a Candida boidinii alcohol oxidase promoter, a Pichia methanolica alcohol utilization gene 1 (AUG1) promoter, a Pichia methanolica alcohol utilization gene 2 (AUG2) promoter, a Pichia methanolica dihydroxyacetone synthase gene promoter, a Pichia methanolica formate dehydrogenase gene promoter, and a Pichia methanolica catalase gene promoter. In a preferred embodiment, the AOX1 promoter is used in Pichia pastoris vectors, and the AUG1 promoter is used in Pichia methanolica vectors.

A cDNA encoding chitin deacetylase is used to construct the recombinant yeast strain for chitin deacetylase expression. In a preferred embodiment said cDNA encoding chitin deacetylase is obtained from Mucor rouxii. The cDNA sequence of Mucor rouxii encoding chitin deacetylase used in the present invention is disclosed in U.S. Pat. No. 6,004,795 (Thireos & Kafetzopoulos). It is noted that the cDNA sequence used in the present invention is the fragment corresponding to the chitin deacetylase coding region (corresponding to the mature protein). The DNA sequence used in the present invention is the sequence disclosed in U.S. Pat. No. 6,004,795 to which for cloning purposes additional sequences were added to the DNA sequence at the 3′ and 5′ end, as explained below.

The chitin deacetylase sequence is amplified by PCR technique with the cDNA as template and primers designed on the basis of the sequence of the fragment and containing appropriate restriction sites for the cloning into the vectors. In a further embodiment, the obtained PCR products are cloned in the vectors that were previously linearized by restriction with appropriate enzymes depending of the used vector (XhoI, NotI, BamHI, Apa I, Sac II, Kpn I). Appropriate restriction sites are provided for cloning in the Pichia expression vectors, which preferably comprise XhoI-NotI for cloning in pPIC9, pPIC9K, pPICZα, pGAPα and pMETα vectors and XhoI-BamHI for cloning in a pHIL-S1 vector. However, it is noted that the use of the XhoI site for cloning requires that the sequence encoding the KEX2 site, which is necessary for an efficient cleavage of the secretion signal peptide from the chitin deacetylase protein, is recreated. For that reason, in another preferred embodiment, additional nucleotide sequences are provided at the 5′ end of chitin deacetylase cDNA sequence, in the expression vector. Preferably, the additional nucleotide sequences encode the amino acid residues Glu-Ala-Glu-Ala in the case where the chitin deacetylase enzyme is expressed with the vectors pPIC9, pPIC9K, pPICZα, pGAPα and pMETα, and the additional nucleotide sequences encode one additional amino acid residue Arg in the case where the chitin deacetylase enzyme is expressed with the pHIL-S1 vector. As a consequence, the sequence of the recombinant chitin deacetylase comprises additional amino acids at the N-terminal end. Thus, the sequence of the recombinant chitin deacetylase expressed in yeast as described in the present invention is slightly different from the sequence of the natural mature chitin deacetylase protein, which is produced by the micro-organism, from which the chitin deacetylase encoding cDNA sequence was used in the present expression vectors. However, advantageously, the additional amino acid residues have no effect on the activity of the recombinant chitin deacetylase and on the stability of the protein.

In another preferred embodiment, additional tag sequences can be provided at the 5′ and/or the 3′ terminal end of the chitin deacetylase cDNA sequence, such that additional amino acids are provided at the N- and/or the C-terminal site respectively of the expressed enzyme. Such tag sequences may be advantageously applied in the purification process of the expressed enzyme. Examples of suitable tag sequences include but are not limited to polyhistidine (6×His) tag, polyarginine-tag, Flag-tag, Strep-tag, c-myc-tag, S-tag, cellulose-binding domain, chitin-binding domain, glutathione S-transferase-tag, maltose-binding protein, and preferably polyhistidine (6×His) tag, polyarginine-tag, FLAG-tag, Strep-tag, c-myc-tag, cellulose-binding domain and glutathione S-transferase-tag, and more preferably polyhistidine (6×His) tag.

In a preferred embodiment, expression and secretion of chitin deacetylase in fusion to a C-terminal tag, like for example a polyhistidine (6×His) tag, can be obtained after cloning of a cDNA encoding chitin deacetylase in a vector like for example pPICZα, pPIC6α, pGAPZα, pPICZα-E, pFLD1α or pMETα (commercially available from Invitrogen). The vectors contain a C-terminal polyhistidine (6×His) tag for rapid purification with metal-chelating resin, and a C-terminal epitope tag for convenient detection with adequate antibodies: c-myc epitope in pPICZα, pPIC6α, pGAPZα and V5 epitope in pFLD1α and pMETα. Particularly preferred vectors for cloning the chitin deacetylase in C-terminal fusion with a (6×His) tag are pPICZα, pGAPZα or pMETα. The vectors can also be used to express chitin deacetylase without the C-terminal peptide when a stop codon is introduced at the end of the chitin deacetylase cDNA sequence. In an example, the chitin deacetylase sequence is cloned in vectors allowing the expression of recombinant protein fused to a C-terminal tag, like polyhistidine (6×His) tag, in order to facilitate the detection and the purification of the recombinant protein. Preferred used vectors are pPICZα, pGAPZα or pMETα (Invitrogen vectors) including a sequence coding for such C-terminal tag. The chitin deacetylase sequence is amplified by PCR technique with the cDNA as template and appropriate designed primers containing restriction sites for the cloning, more preferably XhoI and NotI sites. After amplification, the chitin deacetylase sequence is cloned in the vectors previously linearized by restriction with XhoI and NotI enzymes. The use of the XhoI site requires an additional nucleotide sequence at the 5′ end of the chitin deacetylase cDNA sequence to recreate the Kex2 cleavage site. Thus the sequence of the recombinant chitin deacetylase expressed in Pichia pastoris as described here is different from the sequence of native chitin deacetylase. The additional amino acids at the N-terminal end of the recombinant protein have no negative effect on chitin deacetylase activity and stability.

In another preferred embodiment, expression and secretion of chitin deacetylase in fusion to a N-terminal tag, like for example a polyhistidine (6×HIS) tag, can be obtained after cloning a tag sequence, like for example a (6×CAT) sequence, at the 5′ end of the chitin deacetylase sequence. The cloning can be performed in vectors like pPIC9, pHIL-S1, pPIC9K, pPICZα, pPIC6α, pGAPZα, pPICZα-E, pFLD1α or pMETα. If the used vectors contain a C-terminal tag, like pPICZα, pPIC6α, pGAPZα, pPICZα-E, pFLD1α or pMETα, a stop codon could be introduced at the end of the chitin deacetylase cDNA sequence in order to express the protein without the C-terminal peptide. Particularly preferred vectors for cloning the chitin deacetylase in N-terminal fusion with a (6×CAT) tag are pPIC9, pHIL-S1, pFLD1α, pGAPZα and pPICZα. In an example, an additional nucleotide sequence encoding a polyhistidine (6×His) tag is introduced at the 5′ end of chitin deacetylase cDNA sequence in order to facilitate the detection and the purification of the recombinant protein. Preferred used vectors are pPIC9, pHIL-S1 and pPICZα (Invitrogen vectors). The chitin deacetylase nucleotide sequence is amplified by PCR technique with the cDNA as template and appropriate designed primers. The 5′ primer contains a (6×CAT) sequence preceded by a sequence containing the Kex2 site and the XhoI restriction site. The 3′ primer contains a sequence corresponding to the NotI restriction site and a stop codon at the end of the chitin deacetylase cDNA sequence. After PCR amplification, the (6×CAT)-cDNA sequence is cloned in the pPIC9 vector previously linearized by XhoI-NotI restriction. Thus the sequence of the recombinant chitin deacetylase expressed in Pichia pastoris as described here is different from the sequence of native chitin deacetylase. The addition of a (6×His) tag has no effect on the activity of the recombinant chitin deacetylase.

The vectors used for chitin deacetylase expression preferably comprise a “selectable marker gene”. This selectable marker allows the transformed cells to grow under conditions in which untransformed cells cannot multiply. The selectable marker gene preferably comprises a gene for the selection of yeast transformants such as HIS4, ADE2, ADE1, ARG4, URA3 or genes that provide resistance to antibiotics, such as G418 and other neomycin-type antibiotics (kanamycin resistance gene), hygromycin B (hygromycin B-phosphotransferase gene), aureobasidin A (AUR1 gene), blasticidin (bsd gene), and bleomycin/phleomycin-type antibiotics such as zeocin, as well as ampicillin resistance genes. Preferably, the Pichia vectors carry a selectable marker gene such as HIS4 in pPIC9, pPIC9K and pHIL-S1, ADE2 in pMETα vectors, bsd in pPIC6α, or a zeocin resistance gene in pPICZα, pGAPZα and pFLD1α vectors.

In another preferred embodiment, the used vectors are secretion vectors that carry a secretion signal sequence to direct the transport of the protein to the extracellular medium. The term “secretion signal sequence” refers to a nucleotide sequence coding for a peptide at the N-terminus of the primary translation product that is responsible for directing secretory proteins into the secretion pathway. Examples of suitable secretion signal sequences for use in the present invention comprise but are not limited to a nucleotide sequence coding for the Saccharomyces cerevisiae α-factor prepro peptide, for the Pichia pastoris alkaline phosphatase signal peptide (PHO) or for the PHA-E signal peptide from the plant lectin Phaseolus vulgaris agglutinin (Raemaekers R. J. M. et al., Eur. J. Biochem., 1999, 65, 394-403) to direct transport of the protein to the extracellular medium. In a preferred embodiment, the secretion signal sequence includes the Saccharomyces cerevisiae α-factor prepro signal sequence in pPIC9, pPIC9K, pPIC6α, pGAPZα, pFLD1α, pPICZα and pMETα vectors, or the Pichia pastoris alkaline phosphatase signal sequence in pHIL-S1 vectors.

In a further preferred embodiment, the above-mentioned expression vectors are applied for transformation of yeasts, and preferably Pichia yeasts. Transformation can be performed according to methods known to a person skilled in the art. In a preferred embodiment, according to the way used for the integration of the constructed vector into the yeast genome, different types of transformants can be generated. Transformants may be characterized by Mut⁺ (wild type for Methanol utilization) or Mut^{s} (Methanol utilization slow) phenotype. Mut⁺ and Mut^{s} transformants can be easily distinguished after replica-plating on dextrose and methanol plates. Moreover some integration ways generate multicopy transformants. Another way to obtain multicopy transformants is to use a vector for multicopy integration like for example pPIC9K vector (Invitrogen vector). pPIC9K plasmid contains a kan gene conferring resistance to G418 antibiotic in Pichia. The level of G418 resistance depends on the number of kan genes integrated in the yeast genome. As the kan gene is fused to the gene of interest, the presence of the kan gene on the plasmid can be used to detect pPIC9K transformants harboring multiple copies of the CDA gene. This embodiment is illustrated in example 10. Therefore, in another embodiment, the present invention also relates to a recombinant yeast multicopy strain capable of expressing chitin deacetylase comprising more than one copy of the nucleic acid molecule encoding chitin deacetylase from Mucor rouxii.

Optionally, the different types of obtained transformants can be screened for their ability to express chitin deacetylase when they are cultivated in liquid medium. Chitin deacetylase activity can be measured in the culture supernatant by well-known methods (Kafetzopoulos et al. 1993. PNAS USA, 90, 2564; Araki and Ito, 1975. European Journal of Biochemistry, 55, 71-78). Before screening for enzyme expression, transformants can be also analyzed by PCR to determine if the gene of interest has integrated into the Pichia genome.

In a more preferred embodiment, the present invention provides a recombinant yeast strain, preferably a Pichia strain, capable of expressing chitin deacetylase wherein said recombinant yeast is provided with an expression vector, preferably a Pichia expression vector, that comprises a nucleic acid molecule encoding chitin deacetylase (CDA), an inducible and preferably an alcohol-inducible promoter, a selectable marker sequence, a secretion signal sequence, and optionally additional tag sequences provided at the 5′ and/or 3′ terminal end of nucleic acid molecule encoding CDA. In a particularly preferred embodiment, said recombinant yeast strain comprises a recombinant Pichia pastoris strain capable of expressing chitin deacetylase obtained from Mucor rouxii, wherein said recombinant yeast is provided with an expression vector that comprises the cDNA sequence encoding chitin deacetylase from Mucor rouxii, a methanol-inducible promoter, a selectable marker sequence, a secretion signal sequence and optionally additional (tag) sequences provided at the 5′ and/or 3′ terminal end of nucleic acid molecule encoding CDA. In another preferred embodiment, the present invention provides a recombinant yeast strain which corresponds to a strain as represented in Table 2.

TABLE 2
Non-limiting examples of recombinant yeast strains according to the
present invention

| | | | | Secretion signal | Selectable | N-terminal | C-terminal |
|---|---|---|---|---|---|---|---|
| Strain | vector | promoter | CDA | sequence | marker gene | TAG sequence | TAG sequence |
| P. pastoris | pPIC9 | AOX1 | M. rouxii | S. cerevisiae | HIS4 | — | — |
| GS115, KM71 | | | | α-factor | | | |
| or SMD1168 | | | | | | | |
| P. pastoris | pPIC9 | AOX1 | M. rouxii | S. cerevisiae | HIS4 | 6XHIS | — |
| GS115, KM71 | | | | α-factor | | | |
| or SMD1168 | | | | | | | |
| P. pastoris | pPIC9 | AOX1 | M. rouxii | S. cerevisiae | HIS4 | — | 6XHIS |
| GS115, KM71 | | | | α-factor | | | |
| or SMD1168 | | | | | | | |
| P. pastoris | pPIC9 | AOX1 | M. rouxii | S. cerevisiae | HIS4 | 6XHIS | 6XHIS |
| GS115, KM71 | | | | α-factor | | | |
| or SMD1168 | | | | | | | |
| P. pastoris | pPICZ-α | AOX1 | M. rouxii | S. cerevisiae | Zeocin | — | — |
| GS115, X33, | | | | α-factor | Resistance | | |
| KM71H, | | | | | gene | | |
| SMD1168H | | | | | | | |
| P. pastoris | pPICZ-α | AOX1 | M. rouxii | S. cerevisiae | Zeocin | — | 6XHIS |
| GS115, X33, | | | | α-factor | Resistance | | |
| KM71H, | | | | | gene | | |
| SMD1168H | | | | | | | |
| P. pastoris | pPICZ-α | AOX1 | M. rouxii | S. cerevisiae | Zeocin | 6XHIS | — |
| GS115, X33, | | | | α-factor | Resistance | | |
| KM71H, | | | | | gene | | |
| SMD1168H | | | | | | | |
| P. pastoris | pPICZ-α | AOX1 | M. rouxii | S. cerevisiae | Zeocin | 6XHIS | 6XHIS |
| GS115, X33, | | | | α-factor | Resistance | | |
| KM71H, | | | | | gene | | |
| SMD1168H | | | | | | | |
| P. pastoris | pPIC9K | AOX1 | M. rouxii | S. cerevisiae | HIS4 | — | — |
| GS115, KM71 | | | | α-factor | | | |
| or SMD1168 | | | | | | | |
| P. pastoris | pPIC9K | AOX1 | M. rouxii | S. cerevisiae | HIS4 | 6XHIS | — |
| GS115, KM71 | | | | α-factor | | | |
| or SMD1168 | | | | | | | |
| P. pastoris | pPIC9K | AOX1 | M. rouxii | S. cerevisiae | HIS4 | — | 6XHIS |
| GS115, KM71 | | | | α-factor | | | |
| or SMD1168 | | | | | | | |
| P. pastoris | pPIC9K | AOX1 | M. rouxii | S. cerevisiae | HIS4 | 6XHIS | 6XHIS |
| GS115, KM71 | | | | α-factor | | | |
| or SMD1168 | | | | | | | |
| P. pastoris | pHIL-S1 | AOX1 | M. rouxii | P. pastoris | HIS4 | — | — |
| GS115, KM71 | | | | PHO1 | | | |
| or SMD1168 | | | | | | | |
| P. pastoris | pHIL-S1 | AOX1 | M. rouxii | P. pastoris | HIS4 | 6XHIS | — |
| GS115, KM71 | | | | PHO1 | | | |
| or SMD1168 | | | | | | | |
| P. pastoris | pHIL-S1 | AOX1 | M. rouxii | P. pastoris | HIS4 | — | 6XHIS |
| GS115, KM71 | | | | PHO1 | | | |
| or SMD1168 | | | | | | | |
| P. pastoris | pHIL-S1 | AOX1 | M. rouxii | P. pastoris | HIS4 | 6XHIS | 6XHIS |
| GS115, KM71 | | | | PHO1 | | | |
| or SMD1168 | | | | | | | |
| P. pastoris | pFLD1α | PFLD1 | M. rouxii | S. cerevisiae | Zeocin | — | — |
| GS115, X33, | | | | α-factor | Resistance | | |
| KM71H, | | | | | gene | | |
| SMD1168H | | | | | | | |
| P. pastoris | pFLD1α | PFLD1 | M. rouxii | S. cerevisiae | Zeocin | — | 6XHIS |
| GS115, X33, | | | | α-factor | Resistance | | |
| KM71H, | | | | | gene | | |
| SMD1168H | | | | | | | |
| P. pastoris | pFLD1α | PFLD1 | M. rouxii | S. cerevisiae | Zeocin | 6XHIS | — |
| GS115, X33, | | | | α-factor | Resistance | | |
| KM71H, | | | | | gene | | |
| SMD1168H | | | | | | | |
| P. pastoris | pFLD1α | PFLD1 | M. rouxii | S. cerevisiae | Zeocin | 6XHIS | 6XHIS |
| GS115, X33, | | | | α-factor | Resistance | | |
| KM71H, | | | | | gene | | |
| SMD1168H | | | | | | | |
| P. pastoris | pGAPZα | PGAP | M. rouxii | S. cerevisiae | Zeocin | — | — |
| GS115, X33, | | | | α-factor | Resistance | | |
| KM71H, | | | | | gene | | |
| SMD1168H | | | | | | | |
| P. pastoris | pGAPZα | PGAP | M. rouxii | S. cerevisiae | Zeocin | — | 6XHIS |
| GS115, X33, | | | | α-factor | Resistance | | |
| KM71H, | | | | | gene | | |
| SMD1168H | | | | | | | |
| P. pastoris | pGAPZα | PGAP | M. rouxii | S. cerevisiae | Zeocin | 6XHIS | — |
| GS115, X33, | | | | α-factor | Resistance | | |
| KM71H, | | | | | gene | | |
| SMD1168H | | | | | | | |
| P. pastoris | pGAPZα | PGAP | M. rouxii | S. cerevisiae | Zeocin | 6XHIS | 6XHIS |
| GS115, X33, | | | | α-factor | Resistance | | |
| KM71H, | | | | | gene | | |
| SMD1168H | | | | | | | |

In another particularly preferred embodiment, the invention provides a recombinant yeast strain having accession number MUCL 44353. A recombinant yeast strain obtained according to the present invention has been deposited to the BCCM-MUCL fungi and yeast Collection in the Scientific Institute of Public Health, Louis Pasteur, Brussels, Belgium on Jan. 24, 2003. The deposited strain has the characteristics of the allocated accession number MUCL 44353.

In another aspect the present invention relates to a method of producing chitin deacetylase at high level using a recombinant yeast strain. The present invention provides a method for culturing by fermentation a recombinant yeast strain for expression of chitin deacetylase and the secretion of high levels of the protein. As with most expression systems, the behaviors of particular constructs in a fermentation process can vary and the same protocol cannot be used with all of them. Moreover, gene expression depends on careful control of fermentation conditions. To obtain an optimal control of cell growth and protein expression the choice of suitable parameters therefore is quite difficult. When applying general requirements and technical conditions known in the art for media and fermentation protocols, the production of chitin deacetylase is low, or even absence of chitin deacetylase expression occurs. Therefore, the present application provides an optimization of the prior art fermentation conditions for preparing chitin deacetylase from recombinant yeasts cells. Advantageously, according to the improved method, high levels of secreted chitin deacetylase can be obtained, which in addition is essentially free of any trace activity of chitin or chitosan degrading enzymes, such as chitinases or chitosanases or the like. Moreover, the improved method is simple, faster than currently applied methods and is cost-effective. In a main embodiment, the method according to the present invention for producing chitin deacetylase by a recombinant yeast strain comprises the steps of:

constructing a recombinant yeast strain capable of expressing chitin deacetylase according to the present invention and as explained above by recombinant DNA techniques,

preparing a pre-culture comprising said recombinant yeast strain,

inoculating a suitable amount of said pre-cultured recombinant yeast strain in a suitable fermentation medium and incubating said recombinant yeast strain in said fermentation medium for a suitable period,

feeding for a suitable period of time said incubated recombinant yeast strain with a suitable substrate which controls proliferation of the yeast,

supplementing for a suitable period of time the medium of the incubated recombinant yeast strain with an inducer, capable of stimulating the transcription and translation of the gene encoding chitin deacetylase,

clarifying the medium such that yeast cells are removed from the medium and the supernatant of the medium which comprises chitin deacetylase is retained, and

isolating said chitin deacetylase from said supernatant by chromatographic techniques.

Fermentation of a recombinant chitin deacetylase yeast strain according to this invention can take place in a fermentor vessel. The fermentation process preferably comprises 4 steps: 1) a pre-culture, 2) a batch growth phase, 3) a fed-batch growth phase and 4) an inducer fed-batch phase to induce the expression of the recombinant protein. The present method is based on a fermentation process comprising an initial pre-culture, followed by a growth phase on a carbon source to build up the biomass and an inducer feeding phase to induce the inducible promoter comprised in the expression vector and thus to induce protein expression. The term “batch” refers to a fermentation step that is performed in a fixed volume of medium with a closed system, in which the composition of the medium is determined at the beginning of the fermentation. That is, medium is inoculated with one or more yeast cells at the start of fermentation step, and fermentation is allowed to proceed. Within batch cultures, yeast cells pass through a static lag phase to a high growth log phase, and, finally, to a stationary phase, in which the growth rate is diminished or stopped. The term “fed-batch” refers to a fermentation step that is similar to a typical batch step, except that the substrate for the yeasts is added in increments as the fermentation progresses.

Pre-Culture

In a further embodiment, said recombinant host strain is first pre-cultured in a liquid medium comprising adequate carbon sources, nitrogen sources and nutrients. Preferred pre-culture media comprise YPG or BMGY, which composition is described hereafter. YPG suitably comprises in weight of element per liter of culture medium, 6 g yeast extract, 5 g bacto peptone, 10 g glycerol, and is prepared with deionized water. BMGY suitably comprises in weight of element per liter of culture medium, 10 g yeast extract, 20 g bacto peptone, 10 g glycerol, 13.4 g YNB, 100 mM (pH 6) potassium phosphate, and 0.04 mg biotin, and is prepared with deionized water.

Preferably, the pre-culture medium is inoculated with a colony of the recombinant yeast strain which is obtained from a fresh agar plate or from a frozen glycerol stock. For long-term storage, the recombinant yeast cells are preferably kept in a 15% glycerol solution at −70° C. Another possibility is to inoculate the pre-culture with recombinant yeast cells from a working seed (WS) culture, the WS being inoculated with recombinant yeast cells from frozen glycerol stock.

The pre-culture is preferably grown at a temperature comprised between 25° C. and 35° C. and more preferably grown from 28° C. to 30° C.

Fermentation—Batch Growth Phase

In a further embodiment, a volume of the pre-cultured recombinant yeast is used to inoculate the prepared fermentation medium. The pre-culture volume being inoculated in the fermentor vessel, preferably comprises 3-20% of initial fermentation volume, and more preferably 5-10% of initial fermentation volume. Inoculation of the fermentation medium corresponds to the starting of the batch growth phase. During this phase, the recombinant yeast grows causing, among other modifications, the decrease in the concentration of dissolved oxygen (DO₂) and in the concentration of the carbon source in the fermentation medium, which is preferably glycerol.

The fermentation medium may comprise various carbon sources and/or nutrients suitable for its proliferation. The various carbon sources and/or nutrients to be used for the primary culture of the host cell are known carbon sources and/or nutrients which are suitable for the recombinant yeast cells to be cultured. Examples of carbon energy sources may include glycerol, methanol, glycerine, sorbitol, glucose, fructose, galactose, maltose, maltodextrin and sucrose, which may be used alone or in combination, and examples of the nutrient include nitrogen sources, e.g., yeast extract, bactopeptone, casamino acid, ammonia, ammonium phosphate and ammonium acetate, phosphate sources e.g., phosphoric acid, methylamine and ammonium phosphate, and inorganic sources. The medium may be supplemented with other specific components like for example surfactants (see example 11), and more preferably non-ionic detergent like polyoxyethylenesorbitan monolaurate (Tween), which may facilitate and increase the release of proteins; or metallic ions (see example 12), for example Co⁺⁺, Mg⁺⁺, Mn⁺⁺ or Zn⁺⁺, and more preferably Co⁺⁺ (sulfate or chloride) which has a slight activator effect on the enzyme activity; or inducers of the expression of chitin related enzymes, and more preferably chitin or chitosan (see example 13); or a mixture of the different components (see example 14). Preferably, according to the present invention, the fermentation medium comprises glycerol as a carbon source, and calcium, potassium, phosphate, magnesium, and ammonia.

An illustrative fermentation minimal medium of 61, that can be used for performing a fermentation in a 101 vessel, is prepared by mixing the following compounds: 26.7 ml (0.45 mol/l) of H₃PO₄ 85%, 0.99 g (0.0067 mol/l) of CaCl₂.2H₂O, 18.2 g (0.105 mol/l) of K₂SO₄, 14.9 g (0.06 mol/l) of MgSO₄.7H₂O, and 32 g (0.347 mol/l) of glycerol. The medium is preferably prepared with deionized water and sterilized, e.g. by treating for 20 min at 120° C. It is clear that the composition of the fermentation medium may vary. Those of skill in the art can vary these particular ingredients and amounts. For example, ammonium sulfate can be substituted with ammonium chloride, KOH can be used instead of K₂SO₄, or CaSO₄ can be used instead of CaCl₂.

In an embodiment, the pH of said fermentation medium is adjusted after sterilization of the medium. After preparation and before autoclaving, the pH of the fermentation medium is in a preferred embodiment approximately 1.5. According to the invention, the medium pH is preferably adjusted to a value between 4 and 7 and more preferably between 5.0 and 5.5 after autoclaving and cooling, using the pH probe and regulation system of the fermentor. This operation is critical and the adjustment has to be done at a very slow rate to avoid any precipitate of medium nutrients. If such a precipitate occurs, it has generally negative influence on culture growth and on chitin deacetylase level production.

In a more preferred embodiment, the pH of said fermentation medium is adjusted prior to sterilization of the medium. According to a preferred embodiment, the pH of said fermentation medium is adjusted by a two-stage pH adjustment. A first step comprises an adjustment until pH 3-4 before autoclaving the fermentation medium. The second step comprises progressive and slow adjustment of the pH until a final pH 5.0-5.5 after autoclaving.

According to a preferred embodiment, said pH of the fermentation medium is preferably adjusted with a solution of ammonium hydroxide, preferably comprised between 12 and 40%, and more preferably comprised between 16 and 32%. It is clear that the pH of said fermentation medium could also be adjusted with other solutions as well, for example with a solution of sodium or potassium hydroxide. In this case, the nitrogen source instead of ammonium hydroxide could for example be ammonium sulfate.

When the pH of the fermentation medium has been adjusted, the medium may be supplemented with a solution comprising trace metals such as e.g., iron, zinc, copper, magnesium, manganese, calcium, molybdenum or cobalt, preferably in amount between 0.5 and 5.0 ml per liter medium and more preferred in an amount of 1 ml per liter initial fermentation volume, and comprising vitamins such as e.g., biotin, pantothenic acid, nicotinic acid or thiamine preferably in amount of between 0.5 and 3.0 ml/l of medium and more preferred in an amount of 0.77 ml/l initial fermentation volume. These solutions have preferably been previously sterilized, e.g. by filtration on a 0.22 μm pore-size filter. An illustrative example of a supplementation solution including trace metals comprises MnSO₄.H₂O (1.521 g/l), ZnSO₄.7H₂O (10 g/l), Na₂MoO₄.2H₂O (0.9676 g/l), KI (0.415 g/l), H₂BO₃ (0.0927 g/l), FeCl₃.6H₂O (24.05 g/l), and H₂SO₄ (2 ml/l). Another illustrative example of such supplementation solution including trace metals and vitamins comprises CuSO4.5H₂O (6 g/l), NaI.5H₂O (0.08 g/l), MnSO₄.H₂O (3 g/l), Na₂MoO₄.2H₂O (0.2 g/l), H₃BO₃ (0.02 g/l), CoCl₂.6H₂O (0.5 g/l), ZnCl₂ (20.0 g/l), FeSO₄.7H₂O (65 g/l), Biotin (0.2 g/l), and H₂SO₄ (5.0 ml/l). The supplementation solution and the amount of its particular ingredients may vary. Those of skill in the art can vary these particular ingredients and amounts. For defoaming, a defoaming agent may be added.

The batch growth phase is preferably performed at a temperature comprised between 25° C. and 35° C. and more preferably between 28° C. and 30° C. In another preferred embodiment, the pH value of the fermentation medium during the batch growth phase is maintained at pH between 4 and 7 and more preferably between 5.0 and 5.5. In yet another preferred embodiment, the batch growth phase is performed with vigorous shaking, using a appropriate propeller system, preferably ranging from 250 to 1000 rpm, and more preferably from 400 to 800 rpm, and a sufficient aeration is provided preferably comprised between 0.1 and 3 vvm, and more preferably between 0.5 and 1.5 vvm. Also, the batch growth phase is preferably performed at a pressure of 0.1-0.5 bar above atmospheric pressure. Furthermore, in another preferred embodiment, the batch growth phase is preferably performed at a concentration of dissolved oxygen corresponding to a saturation rate comprised between 20 and 100% and preferably between 30 and 100%, and even more preferred between 30 and 60%. The batch growth phase is maintained until complete consumption of the carbon source, which according to the present invention preferably is glycerol. Complete consumption is indicated by an increase of DO₂. Preferably, the duration of batch growth phase is comprised between 18-24 hours, depending of the density of the initial inoculum.

During this phase, the growth of the recombinant yeast cells can be monitored by measurements of the optical density at 600 nm and by determination of dry cell weight. The final OD₆₀₀ is preferably comprised between 70 and 80, corresponding to a cellular yield preferably comprised between 15 grams dry cells per liter and 30 grams dry cells per liter. Count plate tests can also be performed to evaluate the total density of yeast population and stability of the integrated expression vector. In addition, samples can be harvested during this batch growth phase to analyze the microbiological purity of the culture, protein concentration and chitin deacetylase activity.

Fed-Batch Growth Phase

In another further embodiment, the incubated recombinant yeast is fed for a suitable period of time with a suitable substrate, which controls proliferation of the yeast cell. Once all carbon source comprised in the fermentation medium is consumed from the batch growth phase, the carbon source is fed to the fermentor vessel in order to increase yeast cell biomass under limiting conditions. In a particularly preferred embodiment, the substrate, which controls proliferation of the yeast cell is glycerol.

Preferably, the substrate fed comprises 50% glycerol (w/w) supplemented with acid hydrolyzed casein, e.g. casamino acids. Those of skill in the art can vary these particular ingredients and amounts. In an illustrative example, the solution fed to the fermentor vessel during the fed batch growth phase comprises 1 liter of a 20% casamino acids solution, preferably previously sterilized by autoclaving, and 5 liter of a 50% glycerol feed solution.

The rate of substrate feed delivery, and preferably of glycerol delivery, varies from 5 to 10 ml per hour per liter of fermentation medium, and more preferably from 5 to 8 ml per hour per liter of fermentation medium. As there is a relationship between the rate of substrate delivery in the culture medium and the growth rate of the culture, the fine control of the substrate feed delivery is critical to optimize the culture biomass and thereby the level and the timing of the protein production.

In another preferred embodiment, the operating temperature during the fed-batch phase is comprised between 25° C. and 35° C. and more preferably between 28° C. and 30° C.

Preferably, the pH is maintained during the fed-batch phase to a value comprised between 4 and 7 and more preferably between 5.0 and 5.5. According to a preferred embodiment, said pH medium is preferably adjusted with a solution of ammonium hydroxide, preferably comprised between 12 and 40%, and more preferably comprised between 16 and 32%. It is clear that the pH can also be adjusted with other solutions as well, for example with a solution of sodium or potassium hydroxide. In this case, the nitrogen source instead of ammonium hydroxide could for example be ammonium sulfate.

In another preferred embodiment, the concentration of dissolved oxygen during the fed-batch phase is maintained at a saturation rate comprised between 20 and 100% and preferably between 30 and 100%, and even more preferred between 30 and 60%. Preferably, the duration of fed-batch phase is comprised between 20-30 hours.

During this phase, the growth of the recombinant yeast cells can be monitored with OD₆₀₀ measurements and determination of dry cell weight. The final OD₆₀₀ is preferably comprised between 160 and 200, corresponding to a cellular yield of preferably comprised between 40 grams dry cells per liter and 50 grams dry cells per liter. During this fed batch phase, count plate tests can also be performed to evaluate the total density of yeast population and stability of integrated expression vector. Samples of the culture can also be harvested to analyze microbiological purity of the culture, glycerol concentration, protein concentration and enzyme activity.

Usually there is little or no expression of the protein of interest during this phase due to the absence of an inducer of the inducible promoter controlling the expression of the chitin deacetylase gene in the expression vector of the recombinant host. However, it was found that recombinant yeast cells could express and secrete the protein of interest, in particular chitin deacetylase, when expression is under the control of an inducible promoter, and a substance inducing the promoter was not added to the medium. The weak expression of CDA in absence of an inducer may be due to a weak leaky activity of the inducible promoters.

Inducer Fed-Batch Phase

In another further embodiment, the medium of the cultured yeast is further supplemented for a suitable period of time with an inducer, capable of stimulating the transcription and translation of the gene encoding the chitin deacetylase. However, it will be clear to a person of skill in the art that this step is not required when a recombinant yeast strain is used that shows constitutive expression of the CDA gene. As used herein, the term “inducer” refers to a substance that stimulates the transcription and translation of the chitin deacetylase gene. In the present invention, this inducer preferably is an alcohol and even more preferred is methanol. In a preferred embodiment, when glycerol feeding is stopped and all the glycerol is consumed, a methanol fed-batch feeding phase is started. This phase involves induction of the inducible promoter, preferably the alcohol oxidase promoter, in the recombinant host and expression of the protein of interest.

In a preferred embodiment, the methanol feeding comprises a 100% methanol solution, preferably supplemented with a solution comprising metal trace metals such as e.g., iron, zinc, copper, magnesium, manganese, calcium, molybdenum or cobalt, and vitamins such as e.g., biotin, pantothenic acid, nicotinic acid or thiamine. This solution has preferably been previously sterilized. An illustrative example of such solution may comprise MnSO₄.H₂O (1.521 g/l), ZnSO₄.7H₂O (10 g/l), Na₂MoO₄.2H₂O (0.9676 g/l), KI (0.415 g/l), H₂BO₃ (0.0927 g/l), FeCl₃.6H₂O (24.05 g/l) and H₂SO₄ (2 ml/l). Another illustrative example may comprise CuSO4.5H₂O (6 g/l), NaI.5H₂O (0.08 g/l), MnSO₄.H₂O (3 g/l), Na₂MoO₄.2H₂O (0.2 g/l), H₃BO₃ (0.02 g/l) and CoCl₂.6H₂O. Those of skill in the art can vary these particular ingredients and amounts.

The rate of methanol feeding delivery is critical, especially at the starting of methanol fed-batch feeding phase. Preferably, methanol is introduced slowly to adapt the culture to grow on methanol. If methanol is added too fast, it may kill the yeasts. In a preferred embodiment, the feeding rate for methanol is comprised between 3 ml and 15 ml per hour per liter of fermentation medium, and even more preferred between 3.5 ml and 12 ml per hour per liter of fermentation medium. A fine control of the rate of methanol delivery is essential to obtain a high level of chitin deacetylase production. If the methanol concentration is too low, it leads to starvation. If the methanol concentration is too high, it leads to an accumulation of methanol, eventually to cell death. In both cases, a consequence is a reduced expression of recombinant chitin deacetylase, and possibly the disappearance of protein due to proteolysis, especially when the protein is secreted as in the present invention. In some preferred cases, methanol may be added by steps of various duration.

In another preferred embodiment, the methanol feeding comprises a feeding with a solution comprising glycerol and methanol. In some cases, and more especially with some Mut^{s} strains, which poorly metabolize methanol, a “mixed feed” of glycerol and methanol can be used to induce the expression of the chitin deacetylase protein. Preferably, a slow glycerol feeding is maintained during the first hours of methanol feeding, because it leads to a gradual de-repression of the alcohol oxidase promoter and a better induction of the gene expression. In a preferred embodiment, the feeding rate for glycerol is comprised between 1 ml and 5 ml per hour per liter, and the feeding rate for methanol is comprised between 1 ml and 5 ml per hour per liter.

In another preferred embodiment, the operating temperature during the inducer fed-batch phase is comprised between 25° C. and 35° C. and more preferably between 28° C. and 30° C.

Preferably, the pH is maintained during the inducer fed-batch phase to a value comprised between 4 and 7 and more preferably between 5.0 and 5.5. According to a preferred embodiment, said pH medium is preferably adjusted with a solution of ammonium hydroxide, preferably comprised between 12 and 40%, and more preferably comprised between 16 and 32%. It is clear that the pH can also be adjusted with other solutions as well, for example with a solution of sodium or potassium hydroxide. In this case, the nitrogen source instead of ammonium hydroxide could for example be ammonium sulfate.

In yet another preferred embodiment, the concentration of dissolved oxygen during the inducer fed-batch phase is maintained at a saturation rate comprised between 20 and 100% and preferably between 30 and 100%, and even more preferred between 30 and 60%. Once the culture is adapted to the inducer, it is important to use the DO₂ spike test to analyze the state of the culture, the DO₂ being a sensitive indicator of cell metabolism. Preferably, the duration of the methanol fed-batch phase is comprised between 48 to 120 hours.

In another preferred embodiment, biotin is added every 24 hours during the methanol fed-batch phase, for example 1 ml of a solution of 0.02% biotin per liter of fermentation medium per 24 hours can be added. It will be clear that the rate of biotin addition may vary.

During this phase, the growth of the recombinant yeast cells can be monitored with OD₆₀₀ measurements and determination of dry cell weight. The final OD₆₀₀ is preferably comprised between 220 and 250, corresponding to a cellular yield comprised preferably between 40 grams dry cells per liter and 75 grams dry cells per liter. During this fed batch phase, count plate tests can also be performed to evaluate the total density of yeast population and stability of integrated expression vector. Samples of the culture can also be harvested to analyze microbiological purity of the culture, glycerol concentration, protein concentration and enzyme activity.

Removal of Yeast Cells

In another embodiment the present invention relates to the downstream process following directly the fermentation and preceding the purification of the chitin deacetylase. Preferably, the downstream process is started after a total fermentation time of 120 to 192 hours, and more preferably of 120 to 168 hours. The recovery operations involve two major steps: 1) clarification and 2) concentration of the fermentation medium. The objective of the treatment is to remove cells and to concentrate and equilibrate the supernatant such that it is suitable for use in a further purification step.

In an embodiment, yeast cells are removed from the medium by centrifugation and micro-filtration such that the supernatant of the medium which comprises chitin deacetylase is retained. Since the cDNA encoding chitin deacetylase is preferably cloned in a vector for secreted expression, the chitin deacetylase is produced in the extracellular medium. Therefore, the yeast cells can be removed from the fermentation medium and the supernatant is retained. In a preferred embodiment, the fermentation medium is centrifuged and subsequently filtrated by tangential microfiltration on a 0.22 μm pore-size membrane. As a part of the secreted product can be trapped with the cells, optionally an intermediate step of cell washing can be applied before the microfiltration.

It is noted that small amounts of CDA, that have not been secreted, may remain onto the yeast cells and that these amounts can be retrieved as well, if desired.

In a further preferred embodiment, the obtained supernatant is further concentrated, preferably by tangential ultrafiltration and diafiltration. The concentration step results in decreased volumes as well as higher protein concentration. Smaller volumes are easier to handle in subsequent steps and higher protein concentration minimizes protein losses during purification. Preferably, the obtained supernatant is first concentrated by tangential ultrafiltration on a membrane of preferably 10.000 to 30.000 NMWC (nominal molecular weight cut-off). The range of this distribution is preferably chosen on the basis of the molecular weight of the recombinant chitin deacetylase. The final resulting concentration factor is preferably 10 to 20 times. Following the concentration by ultrafiltration, a step of diafiltration is preferably used to remove salts from the remaining supernatant solution and to equilibrate the supernatant at the correct ionic strength such that the supernatant is suitable for the subsequent purification step. For diafiltration, buffer is added to the concentrated supernatant and ultrafiltration continues until the filtrate reaches a conductivity of preferably 1 ms/cm or less, which generally needs 3-4 cycles of diafiltration. The used buffer preferably is 5 mM sodium succinate at pH 5.5. In a preferred embodiment, the membrane for diafiltration is a membrane of preferably 10.000 to 30.000 NMWC. The final resulting concentration factor is preferably 10-20 times. Advantageously, the filtrated sample can be frozen at −20° C. for storage (for at least 1 year) before purification, without any significant loss of chitin deacetylase activity.

It will be clear to a person of skill in the art that the above-described steps of ultrafiltration and diafiltration are redundant when recombinant yeast strains are used wherein the expression vectors are provided with tag sequences. The presence of a HIS-tag for instance allows the purification of the recombinant protein by affinity on metal-chelating resin in a one-step purification without any required treatment of the supernatant.

Purification

In another embodiment the present invention relates to the purification of chitin deacetylase from the obtained supernatant.

One embodiment of the invention concerns the purification of the fermentation supernatant previously centrifuged, microfiltered, ultrafiltered and equilibrated at the correct ionic strength by diafiltration. The chitin deacetylase is preferably isolated from said supernatant by cation exchange chromatography in such a way that CDA is obtained which is essentially free of any trace activity of chitin or chitosan degrading enzymes, such as chitinases or chitosanases or the like, which could induce hydrolysis of chitin or chitosan, when used in a process of preparing chitosan from chitin. The purification step is critical because fermentation supernatant contains both chitin deacetylase and chitinase activity. For further use of the chitin deacetylase in methods wherein chitin is converted to chitosan, it is essential to completely remove chitinolytic enzyme activity from the enzymatic preparation, in order to avoid the hydrolysis of the chitin and chitosan polysaccharides.

In a preferred embodiment, the purification method used is cation exchange chromatography. The resin, Q Sepharose Fast Flow or Q Sepharose XL (Amersham Pharmacia Biotech), is previously equilibrated, preferably with a 5 mM sodium succinate buffer at pH 5.5. Then, the supernatant is loaded onto the column and after the column is washed with the same buffer. Elution of chitin deacetylase and chitinase from the column can be performed by a linear gradient of NaCl, preferably at a concentration of 0 to 200 mM, in 5 mM sodium succinate buffer at a pH 5.5. Chitin deacetylase activity is detected in the fractions corresponding to a conductivity range of 1.5-9 ms/cm, and chitinase activity in the fractions corresponding to a conductivity range of 13-19 ms/cm. Elution of chitin deacetylase and chitinase preferably is performed step-wise. 5 mM sodium succinate buffer at a pH 5.5 containing NaCl is added until the buffer conductivity is preferably 3-9 ms/cm, and even more preferably 7.5 ms/cm, which corresponds to the chitin deacetylase elution fraction. In a further step, in order to elute chitinase, a 5 mM sodium succinate buffer at a pH 5.5 same buffer containing NaCl may be added until the final conductivity is 15 ms/cm, which corresponds to the chitinase elution fraction.

In another embodiment, the present invention relates to the purification of chitin deacetylase expressed as fusion to a N-terminal tag, as for example a polyhistidine (6×His) tag. This purification can be performed either with crude supernatant or with a supernatant previously centrifuged and microfiltered as described above. The chitin deacetylase is preferably isolated from said supernatant by metal chelate affinity chromatography. The recombinant chitin deacetylase with N-terminal (6×His) tag is purified by affinity chromatography on metal-chelating resin using metal ions including Cu²⁺, Ni²⁺, Zn²⁺ or Co²⁺, such that the chitin deacetylase obtained after purification is pure at homogeneity. In a preferred embodiment, the purification is performed on chelating resin, like for example HisTrap column (Amersham Pharmacia Biotech) loaded with Ni²⁺ or Co²⁺ ions and previously equilibrated in 20 mM phosphate buffer pH 7.4 containing 0.5 M NaCl and 5-10 mM imidazole. Crude culture supernatant is loaded on the column and the column is washed in the same buffer. Flow through and washing are collected for further analysis. Elution of chitin deacetylase is performed in 20 mM phosphate buffer pH 7.4 containing 0.5M NaCl and 20-500 mM imidazole. Chitin deacetylase activity is detected in the first elution fraction. Chitin deacetylase purified with this procedure is homogeneously pure as judged by polyacrylamide gel electrophoresis and by immunodetection on western blot, as illustrated in example 17. Elution of chitin deacetylase can also be performed in other conditions, for example with buffer containing another competitor like histidine, or with a decreasing pH gradient.

Chitin deacetylase activity can be measured by well-known methods (Kafetzopoulos et al. 1993. PNAS USA, 90, 2564; Araki and Ito, 1975. European Journal of Biochemistry, 55, 71-78) either directly in the culture supernatant, in the supernatant after dialysis, or in the supernatant after fast purification on ion exchanger resin. Another method of enzyme assay comprises determining acetic acid released during the incubation of chitin deacetylase with chitinous substrates (Bergmeyer, H. U. 1974. Methods of Enzymatic Analysis, 1, 112-117).

After downstream treatment and purification, the total chitin deacetylase production according to the present invention is preferably comprised between 40 and 400 mg protein per liter of yeast culture, and even more preferred between 60 and 250 mg protein per liter of yeast culture. It is clear that this amount of chitin deacetylase is considerably higher than the amounts, which can be obtained by isolation of native chitin deacetylase from fungal sources. The yield of chitin deacetylase production according to the present invention is 100 to 400 times higher than can be obtained by isolation of native chitin deacetylase from fungal sources.

In yet another aspect, the present invention relates to purified recombinant chitin deacetylase, which is obtainable by the preparation method according to the present invention, wherein a recombinant yeast strain is applied. In a preferred embodiment, said purified recombinant chitin deacetylase has a molecular mass of ˜75 kDa. On SDS polyacrylamide gel, the enzyme band migrates at an apparent size of ˜75 kDa. This observation was confirmed by size exclusion HPLC analysis (Superdex 75 HR 10/30, Amersham Pharmacia Biotech) in non-denaturing conditions. Said recombinant chitin deacetylase is glycosylated and protein deglycosylation under native conditions results in a total loss of enzyme activity.

In another preferred embodiment, said purified recombinant chitin deacetylase is stable in a pH range from 4.0 to 5.0 and the optimum activity is measured at pH 5.0. Enzyme activity is optimal at 60° C. and the recombinant chitin deacetylase exhibits a good thermostability. As an illustrative example, the enzyme retains 100% activity when exposed to a temperature of 50° C. for 20 up to 60 minutes.

By comparison with the native protein, recombinant chitin deacetylase shows a higher activity in presence of acetate, a well-known inhibitor of this enzyme. As an illustrative example, recombinant enzyme retains ˜90% of initial activity in 10 mM acetate and ˜65% in 200 mM acetate after a 6 hour incubation. Cu⁺⁺ strongly inhibits enzyme activity even at low concentration, as for example 1 mM. Co⁺⁺, Mg⁺⁺, Mn⁺⁺ and Zn⁺⁺, at very low concentrations (<10 mM), have a slight activator effect on the enzyme activity.

Recombinant chitin deacetylase obtained according to the present invention hydrolyzes chitinous substrates such as chitohexaose, carboxymethylchitin, glycol chitin, insoluble colloidal chitin and partially deacetylated chitosans. Preferably, recombinant chitin deacetylase, obtained according to the present invention, can be used in a process of chitin or chitosan deacetylation. As an example, the recombinant enzyme can be used to extend the deacetylation of chitosan, from various origins.

Because the obtained chitin deacetylase according to this method is particularly pure, and even totally pure in the case of the chitin deacetylase with N-terminal tag, and does not contain chitinolytic activity, its application allows producing highly deacetylated chitosan, with no loss of molecular weight and no loss of material, and no need for fractionation of the polymer chains. In addition, recombinant chitin deacetylase, obtained according to the present invention, is particularly suitable for use in the preparation of industrial amounts of chitosan, since it can be prepared in large quantities according to the present invention.

### EXAMPLES

In examples 1, 4, 5, 6, 7, 11, 12, 13 and 14 a P. pastoris GS115 strain comprising a pPIC9 expression vector containing the AOX1 promoter, the sequence encoding the S. cerevisiae α-factor prepro peptide; the cDNA encoding CDA from M. rouxii; and a HIS4 selectable marker gene, is applied. Tag sequences were not introduced. This strain is also referred to as GS115/pPIC9-CDA4.

In example 10 a P. pastoris GS115 strain comprising a pPIC9K expression vector containing the AOX1 promoter, the sequence encoding the S. cerevisiae α-factor prepro peptide; the cDNA sequence encoding CDA from M. rouxii; and a HIS4 selectable marker gene, is applied. Tag sequences were not introduced. This strain is also referred to as GS115/pPIC9K-CDA.

In example 15 a P. pastoris GS115 strain comprising a pPICZα expression vector containing the AOX1 promoter, the sequence encoding the S. cerevisiae α-factor prepro peptide; the cDNA sequence encoding CDA from M. rouxii; and a Zeocin resistance gene and a C-term (6×HIS) tag sequence, is applied. This strain is also referred to as GS115/pPICZα-CDA-C(6×HIS).

In example 16 a P. pastoris GS115 strain comprising a pPIC9 expression vector containing the AOX1 promoter; the sequence encoding the S. cerevisiae α-factor prepro peptide; the cDNA sequence encoding CDA from M. rouxii; the HIS4 marker gene; and N-term (6×HIS) tag sequences. This strain is also referred to as GS115/pPIC9-CDA-N(6×HIS).

In examples 18, 20 and 21, A. oryzae strain having a pUT970 expression vector and carrying, the gpdA promoter sequence, the cDNA of CDA gene of Mucor rouxii, the Sh ble resistance gene, and the Ssa secretion signal sequence is applied. This strain is also referred to as Aspergillus oryzae pUT970/2-CDA8′. Examples 20 and 21 illustrate the production of chitin deacetylase by a recombinant fungal strain grown in AMM medium. In example 20 the AMM medium comprised as carbon source, either soluble starch or sucrose, and was supplemented with a trace element solution. In example 21 the AMM medium contained 30 g l⁻¹ glucidex or sucrose, and was supplemented with a carbon source during the growth of the culture.

### Example 1

### Pre-Culture

The following examples illustrate pre-culture of a recombinant yeast strain capable of expressing chitin deacetylase under the control of a suitable inducible promoter according to the invention.

In a first example, the pre-culture comprises 400 ml of YPG medium contained in a two liter shake flask. 1 ml of working seed culture of a recombinant GS115/pPIC9-CDA4 Pichia strain according to the invention is inoculated in the culture and grown at 30° C., 270 rpm orbital shaking speed, for 22 to 24 hours. Cell growth can be monitored by measuring the absorbance at 600 nm (OD₆₀₀). The final OD₆₀₀ of the pre-culture is preferably comprised between 15 and 20.

In a second example, a fermentor of 20 liter containing 14.5 liter of YPG medium and 15 ml of antifoaming was autoclaved during 30 min. After autoclaving, the pH of the broth was adjusted at 5.5 by addition of 32% NH₄OH. The fermentor was inoculated with 8 ml of GS115/pPIC9-CDA4 strain kept in 15% glycerol at −70° C. The pre-culture was grown at 30° C. with controlled pH at 5.5. Dissolved O₂ was maintained at 30% saturation and shaking in the range 200-800 rpm. After 27 hours (final OD₆₀₀=8.5), the total volume of pre-culture was used to inoculate a fermentor vessel of 200 liter containing 150 liter of minimal medium.

### Example 2

### Method for Preparing a Sample for Monitoring CDA Activity

This example illustrates a rapid and simple method for measuring chitin deacetylase activity in a sample. This method can be applied at any step in the fermentation process according to the present invention for monitoring CDA activity during the preparing process. As an illustrative example, chitin deacetylase activity is determined as described below in a supernatant obtained at the end of an inducer fed-batch phase.

In this example, 2 ml of fermentation supernatant is dialyzed against 2 liter of 5 mM sodium succinate buffer/pH 5.5. The final conductivity of the dialyzed sample preferably is ˜1 ms/cm. The sample can then be applied on a Q Sepharose Fast Flow column (1 ml resin) previously equlibrated in 5 mM sodium succinate buffer/pH 5.5. The column is preferably washed with five column volumes of the same buffer. Chitin deacetylase is then eluted with 5 mM sodium succinate buffer/pH 5.5 containing NaCl such that the final conductivity of the buffer is preferably 7.5 ms/cm. Chitin deacetylase activity can be detected in the first 3 ml of elution volume. Chitin deacetylase activity is measured by well-known methods (see above).

### Example 3

### Purification of Chitin Deacetylase

This example illustrates a purification process according to the present invention for obtaining chitin deacetylase which is essentially free of any trace chitin or chitosan degrading enzymatic activity caused for instance by chitinases, chitosanases or the like.

In this example, a culture medium from a 10-liter fermentor was harvested after 126 hours of fermentation. The sample was centrifuged, microfiltrated, concentrated by ultrafiltration and equilibrated in sodium succinate buffer 5 mM/pH 5.5 by diafiltration as described in the present invention. The final conductivity of the sample was 1.19 ms/cm and the protein content was 0.70 mg/ml.

Chitin deacetylase activity was measured using as substrate partially O-hydroxyethylated chitin (glycol chitin) radio-labeled in N-acetyl groups. The substrate was synthesized as described in Araki and Ito (European Journal of Biochemistry, 55, pp 71-78, 1975). The final total activity of the radio-labeled substrate was adjusted to 100 000 cpm per 5 μl of glycolchitin solution. The enzyme activity determination was performed as described in Araki and Ito (1975), and in Kafetzopoulos et al. (Proceedings of the National Academy of Sciences of the United States of America, 90, pp 2564-2568, 1993) using 5 μl of radio-labeled substrate (corresponding to a total of 100 000 cpm), 20 μl of sample and 25 μl of adequate buffer. It is noted that the same above-described protocol is used throughout the application to measure CDA activity and that chitin deacetylase activity is expressed throughout this application in the unit cpm/ml supernatant. All enzymatic radiometric assays were performed in identical conditions throughout the application. In these conditions, measurement of released acetic acid, expressed in cpm, is proportional to the chitin deacetylase activity and all results as presented in the present application can be compared.

Chitin deacetylase activity in the sample was determined and comprised 6.5 10⁴ cpm/ml. For further purification, the sample (7.5 liter) was loaded on a Q Sepharose XL column (350 ml, Amersham Pharmacia Biotech) equilibrated in the same buffer. After the column was washed, a step-wise elution was performed with 5 mM sodium succinate/pH 5.5 buffer containing NaCl such that the final conductivity was 7.5 ms/cm. The eluted fractions corresponding to a conductivity range of 2.2-7.5 ms/cm were collected and analyzed for chitin deacetylase activity. The total collected volume comprised 335 ml and the protein content was 5.62 mg/ml. The chitin deacetylase activity comprised 44.5 10⁴ cpm/ml and the specific activity was 7.9 10⁴ cpm/mg protein.

### Example 4

### Production of Chitin Deacetylase in a Fermentor Vessel of 15 Liter

This example illustrates the production of chitin deacetylase according to the present invention by using a recombinant Mut^{s} Pichia strain grown in minimal medium supplemented with trace metal solution comprising KI, MnSO₄.H₂O, Na₂MoO₄.2H₂O, H₃BO₃, FeCl₃.6H₂O and ZnSO₄.7H₂O, in mixed feeding methanol-glycerol mode at the start of the production.

A Pichia pastoris GS115/pPIC9-CDA4 pre-culture was grown in two shake flasks of 2 liter containing 0.4 liter of YPG medium. Each flask was inoculated with 250 μl of a strain kept in 15% glycerol at −70° C. After 22 hours (OD₆₀₀=11.2), a fermentor of 15 liter containing 7.2 liter of minimal medium was inoculated with 0.4 l of the pre-culture. The minimal medium consisted of 0.45 mol/l of H₃PO₄ 85%, 0.0067 mol/l of CaCl₂.2H₂O, 0.105 mol/l of K₂SO₄, 0.06 mol/l of MgSO₄.7H₂O, and 0.347 mol/l of glycerol. The medium was prepared with deionized water and sterilized. The pH of the medium was adjusted at 4.5 with 16% NH₄OH before autoclaving, and at pH 5.0 after sterilization and cooling. Then the medium was supplemented with 61 ml of 0.02% biotin and 8 ml of trace metal solution comprising KI, MnSO₄.H₂O, Na₂MoO₄.2H₂O, H₃BO₃ and FeCl₃.6H₂O previously sterilized by filtration on a 0.22 μm pore-size filter. Antifoaming was also added to the medium before sterilization. The fermentation culture was grown at 30° C. with pH maintained at 5.0. Dissolved O₂ was maintained above 30% saturation and vigorously shaking at 200 to 600 rpm. Growth was continued until the glycerol carbon source was exhausted. After 24 hours (OD₆₀₀=17.8), a limited glycerol feed (50% glycerol and 10% casamino acids) was initiated at a rate of 30 ml per hour for the next 28 hours (52 hours elapsed fermentation time, EFT). After this time (OD₆₀₀=50), a glycerol feed (12.5% glycerol, 12.5% casamino acids, 1.7% biotin) was maintained using a stepped rate during 1 hour (3 steps). At the same time, a slow feed of 100% methanol supplemented with 1.69% trace metal solution comprising KI, MnSO₄.H₂O, Na₂MoO₄.2H₂O, H₃BO₃, FeCl₃.6H₂O, and ZnSO₄.7H₂O was started. After 2.5 hours, the methanol feed rate was progressively increased for the remaining fermentation time. A total of 1.704 liter of methanol and 203 grams of glycerol were delivered during methanol fed batch phase. The fermentation ended at 142 hours EFT. The total biomass was estimated to 29 grams dry cell per liter.

The kinetics of chitin deacetylase production showed a latent period during the first 30 hours of induction. At the end of the preparation process, chitin deacetylase activity was estimated to 6.9 10³ cpm per ml of supernatant, measured with a method using radiolabeled glycol chitin as substrate, and the protein content was 2.5 mg per ml of supernatant.

The fermentation medium was then centrifuged to discard the yeast cells and the supernatant was concentrated by ultrafiltration and diafiltered for further purification. In this example, the yield of chitin deacetylase production was estimated by different methods and comprised 190 milligrams of protein per liter of fermentor and the specific activity was 5.86 10⁴ cpm per mg of protein.

### Example 5

### Production of Chitin Deacetylase in a Fermentor Vessel of 22 Liter

This example illustrates the production of chitin deacetylase according to the present invention by using a recombinant Mut^{s} Pichia strain grown in minimal medium supplemented with trace metal solution comprising KI, MnSO₄.H₂O, Na₂MoO₄.2H₂O, H₃BO₃, FeCl₃.6H₂O, and ZnSO₄.7H₂O without mixed feeding methanol-glycerol but with a slow methanol feed rate delivery.

A Pichia pastoris GS115/pPIC9-CDA4 pre-culture was grown in three shaken flasks of 2 l. containing 0.53 l of YPG medium. Each flask was inoculated with 400 μl of a strain kept in 15% glycerol at −70° C. After 21 hours (OD₆₀₀=8.3), a fermentor of 22 liter containing 14.4 liter of minimal medium was inoculated with the total volume of pre-culture (3×0.53 liter). The minimal medium consisted of 0.45 mol/l of H₃PO₄ 85%, 0.0067 mol/l of CaCl₂.2H₂O, 0.105 mol/l of K₂SO₄, 0.06 mol/l of MgSO₄.7H₂O, and 0.347 mol/l of glycerol. The pH of the medium was adjusted to 4.5 with 16% NH₄OH before autoclaving, and to pH 5.5 after sterilization and cooling. Then the medium was supplemented with 122 ml of 0.02% biotin and 16 ml of trace metal solution comprising KI, MnSO₄.H₂O, ZnSO₄.7H₂O, Na₂MoO₄.2H₂O, H₃BO₃, and FeCl₃.6H₂O previously sterilized by filtration on a 0.22 μm pore-size filter. The fermentation culture was grown at 30° C. with controlled pH at 5.5. Dissolved O₂ was maintained above 60% saturation and shaking in the range of 300-1200 rpm. Growth was continued until the glycerol was exhausted. After 24 hours (OD₆₀₀=66.05), a limited glycerol feed (50% glycerol and 10% casamino acids) was initiated at a rate of 62.5 ml per hour for the next 26 hours (50 hours elapsed fermentation time, EFT). At the end of glycerol fed batch phase (OD₆₀₀=129), methanol feeding was slowly started at a rate of 6.6 ml/min with a rate delivery limited to 10% during the first 4 hours (158 ml methanol), to 18% for the next 20 hours and to 10-15% for the remainder of the preparation process. Methanol feeding was supplemented with 6.75 ml of trace metal solution comprising KI, MnSO₄.H₂O, ZnSO₄.7H₂O, Na₂MoO₄.2H₂O, H₃BO₃, and FeCl₃.6H₂O per liter of methanol feed. A chitin deacetylase activity was measured in the supernatant early after the start of methanol feeding (from 20 hours) without lag period. A total of 4.65 liter of methanol was consumed. In this example, the fermentation ended after 143 hours EFT. Culture OD₆₀₀ was 210 and the total biomass was 37 grams of dry cells per liter of fermentation. Chitin deacetylase activity was estimated to 6.5 10³ cpm per ml supernatant. The medium was then centrifuged, concentrated by ultrafiltration and equilibrated by diafiltration. In this example, the yield of chitin deacetylase production as estimated by different methods was 110 milligrams of protein per liter of fermentor and the specific activity was 12.3 10⁴ cpm per mg of protein.

### Example 6

### Production of Chitin Deacetylase in a Fermentor Vessel of 200 Liter

This example illustrates the production chitin deacetylase according to the present invention in a fermentor vessel of 200 liter.

The used strain in this example was Pichia pastoris GS115/pPIC9-CDA4 and the culture was grown in minimal medium supplemented with trace metal solution comprising KI, MnSO₄.H₂O, ZnSO₄.7H₂O, Na₂MoO₄.2H₂O, H₃BO₃, and FeCl₃.6H₂O. General conditions of this preparation were the same as described in example 5, except the volumes that were adapted. The duration and OD₆₀₀ of the different fermentation phases are summarized in the table below.

| Fermentation phase | Duration (hours) | OD₆₀₀ |
|---|---|---|
| Pre-culture | 27 | 8.5 |
| Glycerol Batch growth phase | 18 | 51 |
| Glycerol Fed Batch growth phase | 24.5 | 134 |
| Methanol Fed Batch | 48 | 189 |

A total of 25 liter methanol was consumed. Chitin deacetylase activity was estimated to 15.8 10³ cpm per ml supernatant. The medium was then centrifuged, concentrated by ultrafiltration and equilibrated by diafiltration. The yield of chitin deacetylase production as estimated by different methods was 178 milligrams of protein per liter of fermentor and the specific activity was 26.7 10⁴ cpm per mg of protein.

### Example 7

### Production of Chitin Deacetylase in a Fermentor Vessel of 10 Liter

This example illustrates the production of chitin deacetylase according to the present invention in a fermentor vessel of 10 liter.

Pichia pastoris GS115/pPIC9-CDA4 strain was grown in minimal medium supplemented with trace metal solution comprising KI, MnSO₄.H₂O, ZnSO₄.7H₂O, Na₂MoO₄.2H₂O, H₃BO₃, and FeCl₃.6H₂O. The fermentor was inoculated with 1 liter inoculum grown in flasks shaken at 30° C., 270 rpm. As P. pastoris culture has a high oxygen requirement, the instruction for backpressure during the preparation process was increased and maintained to 0.4 bars to avoid a limitation of the dissolved O₂ during the culture growth. After 23 hours glycerol batch culture, a 50% glycerol feeding was initiated and maintained during 33.5 hours. At the end of fed batch glycerol, the culture reached a OD₆₀₀ of 187. Then methanol feeding was progressively initiated and maintained during 126 hours. A total of 2.2 liter methanol was consumed. The fermentation ended after 183 hours EFT. Culture OD₆₀₀ was 188. In this example, chitin deacetylase activity comprised 12.7 10⁴ cpm per ml supernatant. The medium was then centrifuged, concentrated by ultrafiltration and equilibrated by diafiltration. The yield of chitin deacetylase production as estimated by different methods was 370 milligrams of protein per liter of fermentor in this example, and the specific activity was 37.5 10⁴ cpm per mg of protein.

### Example 8

### Purification of Chitin Deacetylase without Separation of the Fraction Containing Chitinase Activity

The present example illustrates the purification of chitin deacetylase from the supernatant obtained by a method according to the present invention, without separating the fraction having chitinase activity from the supernatant. Supernatant was obtained from a fermentation process as described herein in a 200 liter fermentor. After fermentation, the supernatant was collected by microfiltration (0.45 and 0.22 μm pore-size filters) and centrifugation, then concentrated by ultrafiltration and equilibrated in 5 mM sodium succinate buffer/pH 5.5 by diafiltration. A concentration factor of 25 times was obtained.

A sample of 800 ml, corresponding to an initial fermentation volume of 20 liter, of concentrated supernatant was applied onto a Q Sepharose XL column (350 ml) previously equilibrated with a 5 mM sodium succinate buffer/pH 5.5. The column was washed with this buffer and the elution was performed with a step gradient of the buffer/150 mM NaCl (conductivity 27 ms/cm) at a flow rate of 50 ml per minute. All fractions (23×15 ml) containing chitin deacetylase activity were pooled. In this example, the total protein content of the enzymatic preparation was 6.3 grams and total chitin deacetylase activity was 1.4 10⁸ cpm (radiometric assay), corresponding to a specific activity of 2.2 10⁴ cpm/mg of protein.

This preparation was reacted with partially deacetylated chitin, i.e. 25% acetylated chitosan during 47 hours in pH and temperature conditions compatible with chitin deacetylase activity. Kinetics of deacetylation were monitored by determining acetic acid released from the substrate and viscosity at 37° C. of the reaction mixture was measured in a viscosimeter. Results showed a severe decrease of the viscosity during-incubation of the substrate: 45% after 2 hours, 65% after 24 hours and more than 70% after 47 hours. Such a decrease corresponded to a degradation of the polymer resulting from the presence of chitinolytic activity in the enzymatic preparation.

Thus, the use of a chitin deacetylase purified as described in this example in a process for converting chitin to chitosan induced degradation of the chitosan. Hydrolysis of the chitosan polymers was observed, and chitosan having reduced polymer length and reduced viscosity was obtained.

### Example 9

### Preparation of Chitin Deacetylase with Separation of the Fraction Containing Chitinase Activity

This example illustrates the purification of chitin deacetylase from the supernatant obtained by a method according to the present invention, with separation of the fractions having chitinase activity from the supernatant. The supernatant used in this example was similar to the supernatant used in example 8. 200 ml of concentrated supernatant, corresponding to an initial fermentation volume of 5 liter, was loaded on Q Sepharose XL column (350 ml) previously equilibrated in 5 mM sodium succinate buffer/pH 5.5. The column was washed with this buffer and subsequently developed with a linear gradient of NaCl (0-500 mM) in the same buffer, at a flow rate of 10 ml/minute. Fractions (15 ml) were analyzed for chitin deacetylase and chitinase activities. In this example, analyses showed that chitin deacetylase was mainly eluted in the fractions corresponding to a conductivity range of 2-10 ms/cm, and chitinase activity in the fractions eluted at a conductivity >10 ms/cm. A small part of chitin deacetylase activity was also found in those fractions. On the basis of the analysis and of chromatographic profile, five elution pools were obtained and analyzed for their protein content, chitin deacetylase and chitinase activities, and electrophoretical pattern.

The five enzymatic preparations were reacted with partially deacetylated chitin, 25% deacetylated chitosan during 48 hours and the viscosity of reaction mixtures was monitored (measured at 37° C. with a viscosimeter). In case of enzyme preparations corresponding to an elution range <10 ms/cm, no viscosity decrease was measured in reaction mixtures, even after prolonged time. In contrast, a severe decrease of the viscosity, up to 90% loss in viscosity after 48 hours, was measured in mixtures with enzyme preparations corresponding to an elution range >10 ms/cm. Such a viscosity decrease was also observed with crude fermentation supernatant.

In conclusion, this example illustrates that degradation of chitosan only occur when applying enzymatic preparations which have been eluted in the presence of a NaCl concentration corresponding to a conductivity of >10 ms/cm. This indicates that by careful elution in the presence of a NaCl concentration having a well-defined conductivity range, chitin deacetylase enzymatic preparations can be obtained which are essentially free of chitinase activity.

### Example 10

### Expression of Chitin Deacetylase in pPIC9K/CDA Transformants

This example is an illustration of chitin deacetylase expression from recombinant yeast strains obtained by transformation of Pichia pastoris GS115 strain with pPIC9K/CDA plasmid generating multiple copy integration of the gene of interest. P. pastoris GS115/pPIC9K-CDA previously linearized by restriction with Bgl II for replacement at AOX1 locus, generating thus Mut^{s} strains (named B/Mut^{s} strains) and Sac I for insertion at AOX1 locus, generating thus Mut⁺ strains (named Sc/Mut⁺ strains), or Sal I for insertion at HIS4 locus, generating thus Mut⁺ strains (named Sl/Mut⁺ strains). Transformants were screened on YPD plates containing various concentrations of G418 antibiotic (0.25, 3 and 4 mg/ml). The used nomenclature to identify each strain is the following: plasmid type/insert-restriction site used for plasmid linearization in yeast transformation (B=Bgl II; Sl=Sal I; Sc=Sac I)-clone number-G418 concentration used for the selection (in mg/ml)/strain phenotype (Mut^{s} or Mut⁺).

Pre-cultures were grown in 250 ml YPG medium in 1 liter flasks. Each flask was inoculated with 175 μl of yeast strain kept in 15% glycerol at −70° C. After 17 hours, flasks of 2 liter containing 400 ml of minimal medium (containing only 0.25% of each component by comparison with the same medium used in fermentation) were inoculated with 40 ml of preculture. The pH of the medium was adjusted at 4.5 with 16% NH₄OH before autoclaving, and at pH 5.5 after sterilization and cooling. Then the medium was supplemented with 7.5 ml of 0.02% biotin and 1 ml of trace metal solution 1 (KI, MnSO₄.H₂O, ZnSO₄.7H₂O, Na₂MoO₄.2H₂O, H₃BO₃, FeCl₃.6H₂O) previously sterilized by filtration on 0.22 μm pore-size filter. The cultures were grown at 30° C. with orbital shaking at 270 rpm. After 24 hours of growth, the induction was started by addition of 2.2 ml of 100% methanol. The medium was supplemented by addition of 20 ml of 20% casamino acids, 700 μl of 20 mg/ml biotin (20 mg/ml), 50 μl of trace metal solution 1 (see above) and 6.3 ml of 70% glycerol. After 48 hours of growth, the medium was supplemented every 24 hours with 2.2 ml of 100% methanol, 2 ml of 20% casamino acids, 70 μl of 20 mg/ml biotin, 5 μl of trace metal solution 1 (see above) and 3.2 ml of 70% glycerol. The induction phase was maintained during a total of 96 or 120 hours. Samples of each culture were harvested every 24 hours and chitin deacetylase activity was measured in culture supernatants. Chitin deacetylase activity was measured by radiolabelled method using ³H-glycolchitin and expressed as cpm per ml of culture supernatant. The results are summarized in the table below.

| | 24 hours | 48 hours | 72 hours | 96 hours | 120 hours |
|---|---|---|---|---|---|
| Strain | induction | induction | induction | induction | induction |
| PPIC9K/CDA-B-1-0.25/Mut^{S} | 112 | 1310 | 1526 | 2510 | 2991 |
| PPIC9K/CDA-B-9-3/Mut^{S} | 847 | 1958 | 2927 | 3896 | 4350 |
| PPIC9K/CDA-B-3-4/Mut^{S} | 367 | 1764 | 2630 | 3921 | 4428 |
| PPIC9K/CDA-SI-2-0.25/Mut⁺ | 779 | 1123 | 1535 | 3178 | 4331 |
| PPIC9K/CDA-SI-13-3/Mut⁺ | 1093 | 3232 | 4649 | 4662 | 5214 |
| PPIC9K/CDA-SI-7-4/Mut⁺ | 426 | 488 | 1056 | 1180 | 899 |
| PPIC9K/CDA-Sc-3-4/Mut⁺ | ND | 252 | 1027 | 1907 | ND |

ND: not determined

### Example 11

### Chitin Deacetylase Expression in Medium Supplemented with a Detergent

This example illustrates the preparation of recombinant chitin deacetylase in minimal medium supplemented with a non-ionic detergent like Tween 20 which is polyoxyethylenesorbitan monolaurate. Pichia pastoris GS115/pPIC9-CDA4 pre-culture was grown in a shake flask of 1 liter containing 250 ml of YPG medium. The flask was inoculated with 180 μl of a yeast strain kept in 15% glycerol at −70° C. After 22 h30, 3 flasks of 2 liter containing 400 ml of minimal medium (containing only 0.25% of each component by comparison with the same medium used in fermentation) were inoculated with 45 ml of pre-culture. The pH of the medium was adjusted at 4.5 with 16% NH₄OH before autoclaving, and at pH 5.5 after sterilization and cooling. Then the medium was supplemented with 7.5 ml of 0.02% biotin and 1 ml of trace metal solution 1 (KI, MnSO₄.H₂O, ZnSO₄.7H₂O, Na₂MoO₄.2H₂O, H₃BO₃, FeCl₃.6H₂O) previously sterilized by filtration on a 0.22 μm pore-size filter. The cultures were grown at 30° C. with orbital shaking at 270 rpm. Induction was started by addition of 2.2 ml of 100% methanol. The medium was supplemented by addition of 20 ml of 20% casamino acids, 1.5 ml of 20 mg/ml biotin, 200 μl of trace metal solution 1 (see above) and 6.3 ml of 70% glycerol. In 2 flasks, the medium was also supplemented with a solution of Tween (100 g/l): 0.45 ml in the flask E3 and 4.5 ml in the flask E4. A control flask (E1) without Tween in the culture medium was performed with the same strain in the same conditions as described. After 48 hours of growth, the medium of each flask was supplemented every 24 hours with 2.2 ml of 100% methanol, 2 ml of 20% casamino acids, 1.5 ml of 20 mg/ml biotin, 200 μl of trace metal solution 1 (see above) and 1 ml of 70% glycerol. The induction phase was maintained during a total of 96 hours. Samples of each culture were harvested every 24 hours and chitin deacetylase activity was measured in culture supernatants. Chitin deacetylase activity was measured by radiolabelled method using ³H-glycolchitin and expressed as cpm per ml of culture supernatant. The results are summarized in the table below.

This example illustrates the positive influence of detergent such as tween on CDA production, especially at early time of induction. In culture supplemented with 10% tween, the level of CDA activity is 2.4 times higher after 24 hours of induction in comparison with control culture without detergent.

| | 24 hours | 48 hours | 72 hours | 96 hours |
|---|---|---|---|---|
| Flask | induction | induction | induction | induction |
| E1 | 682 | 3840 | 8897 | 8432 |
| E3 | 825 | 4180 | 5915 | 6785 |
| E4 | 1642 | 4715 | 5850 | 6635 |

### Example 12

### Chitin Deacetylase Expression in Medium Supplemented with Cobalt

This example illustrates the preparation of recombinant chitin deacetylase in minimal medium supplemented with cobalt sulfate at various concentrations. Pichia pastoris GS115/pPIC9-CDA4 preculture was grown in a shake flask of 2 liter containing 300 ml of YPG medium. The flask was inoculated with 200 μl of a yeast strain kept in 15% glycerol at −70° C. After 22.5H, 4 flasks of 2 liter containing each 400 ml of minimal medium (containing only 0.25% of each component by comparison with the same medium used in fermentation) were inoculated with 40 ml of preculture. The pH of the medium was adjusted at 4.5 with 16% NH₄OH before autoclaving, and at pH 5.5 after sterilization and cooling. Then the medium was supplemented with 7.5 ml of 0.02% biotin and 1 ml of trace metal solution 1 (KI, MnSO₄.H₂O, ZnSO₄.7H₂O, Na₂MoO₄.2H₂O, H₃BO₃, FeCl₃.6H₂O) previously sterilized by filtration on a 0.22 μm pore-size filter. The cultures were grown at 30° C. with orbital shaking at 270 rpm. After 24.5H of growth, the induction was started by addition of 2.2 ml of 100% methanol. The medium was supplemented by addition of 20 ml of 20% casamino acids, 750 μl of 20 mg/ml biotin, 100 μl of trace metal solution 1 (see above) and 6.3 ml of 70% glycerol. In 3 flasks, the medium was also supplemented with a solution of 0.1% cobalt sulfate: 1.1 ml in the flask A2, 2.2 ml in the flask A3 and 3.3 ml in the flask A4. A control flask (A1) without cobalt sulfate in the culture medium was performed with the same strain in the same conditions as described. After 48 hours of growth, the medium of each flask was supplemented every 24 hours with 2.2 ml of 100% methanol, 2 ml of 20% casamino acids 20%, 1.5 ml of 20 mg/ml biotin, 200 μl of trace metal solution 1 (see above) and 3.2 ml of 70% glycerol. The induction phase was maintained during a total of 96 hours. Samples of each culture were harvested every 24 hours and chitin deacetylase activity was measured in culture supernatants. Chitin deacetylase activity was measured by radiolabelled method using ³H-glycolchitin and expressed as cpm per ml of culture supernatant. The results are summarized in the table below.

| | 24 hours | 48 hours | 72 hours | 96 hours |
|---|---|---|---|---|
| Flask | induction | induction | induction | induction |
| A1 | 732 | 755 | 1070 | 1260 |
| A2 | 815 | 907 | 1917 | 1722 |
| A3 | 607 | 1057 | 2737 | 2790 |
| A4 | 940 | 1007 | 3370 | 3072 |

Medium supplementation with cobalt increased CDA activity level: up to 3 times higher after 72 hours in comparison with control culture without cobalt.

### Example 13

### Chitin Deacetylase Expression in Medium Supplemented with Chitin or Chitosan

This example illustrates the preparation of recombinant chitin deacetylase in minimal medium supplemented with chitin or chitosan. Pichia pastoris GS115/pPIC9-CDA4 preculture was grown in a shake flask of 2 liter containing 400 ml of YPG medium. The flask was inoculated with 200 μl of a strain kept in 15% glycerol at −70° C. After 24H, 3 flasks of 2 liter containing each 400 ml of minimal medium (containing only 0.25% of each component by comparison with the same medium used in fermentation) were inoculated with 40 ml of preculture. The pH of the medium was adjusted at 4.5 with 16% NH₄OH before autoclaving, and at pH 5.5 after sterilization and cooling. Then the medium was supplemented with 7.5 ml of 0.02% biotin and 1 ml of trace metal solution 1 (KI, MnSO₄.H₂O, ZnSO₄.7H₂O, Na₂MoO₄.2H₂O, H₃BO₃, FeCl₃.6H₂O) previously sterilized by filtration on a 0.22 μm pore-size filter. The cultures were grown at 30° C. with orbital shaking at 270 rpm. After 24.5H of growth, the induction was started by addition of 2.2 ml of 100% methanol. The medium was supplemented by addition of 22 ml of 20% casamino acids and 9 ml of 50% glycerol. In one flask (FE1), the medium was also supplemented with 8 g of crab chitin in powder (Sigma) previously autoclaved in 40 ml water at 100° C. during 10 min. In a second flask (FE2), the medium was supplemented with 4 g of crab chitosan in powder (Sigma) previously autoclaved in 40 ml water at 100° C. during 10 min. A control flask (FE4) without chitin or chitosan in the culture medium was performed with the same strain in the same conditions as described. After 48 hours of growth, the medium of each flask was supplemented every 24 hours with 2.2 ml of 100% methanol. The induction phase was maintained during a total of 96 hours. Samples of each culture were harvested every 24 hours and chitin deacetylase activity was measured in culture supernatants. Chitin deacetylase activity was measured by radiolabelled method using ³H-glycolchitin and expressed as cpm per ml of culture supernatant. The results are summarized in the table below.

The supplementation of the culture medium with chitin or chitosan increased the level of CDA activity. In comparison with the control culture, the CDA activity was 2 to 5 times higher in presence of 2% chitin, and 1.5 to 4 times higher in presence of 1% chitosan.

| | 24 hours | 30 hours | 48 hours | 72 hours | 96 hours |
|---|---|---|---|---|---|
| Flask | induction | induction | induction | induction | induction |
| FE1 | 262 | 230 | 5422 | 4437 | 5115 |
| FE2 | 500 | 1090 | 3560 | 4257 | 4330 |
| FE4 | 292 | 275 | 1150 | 2790 | 2687 |

### Example 14

### Chitin Deacetylase Expression in Medium Supplemented with Cobalt and Chitin

This example illustrates the preparation of recombinant chitin deacetylase in minimal medium supplemented with cobalt sulfate and chitin. Pichia pastoris GS115/pPIC9-CDA4 pre-culture was grown in a shake flask of 2 liter containing 300 ml of YPG medium. The flask was inoculated with 200 μl of a yeast strain kept in 15% glycerol at −70° C. After 22.5H, 2 flasks of 2 liter containing each 400 ml of minimal medium (containing only 0.25% of each component by comparison with the same medium used in fermentation) were inoculated with 40 ml of pre-culture. The pH of the medium was adjusted at 4.5 with 16% NH₄OH before autoclaving, and at pH 5.5 after sterilization and cooling. Then the medium was supplemented with 7.5 ml of 0.02% biotin and 1 ml of trace metal solution 1 (KI, MnSO₄.H₂O, ZnSO₄.7H₂O, Na₂MoO₄.2H₂O, H₃BO₃, FeCl₃.6H₂O) previously sterilized by filtration on a 0.22 μm pore-size filter. The cultures were grown at 30° C. with orbital shaking at 270 rpm. After 24.5H of growth, the induction was started by addition of 2.2 ml of 100% methanol. The medium was supplemented by addition of 20 ml of 20% casamino acids, 750 μl of 20 mg/ml biotin, 100 μl of trace metal solution 1 (see above) and 6.3 ml of 70% glycerol. In one flask (A5), the medium was also supplemented with 2.2 ml of 1 g/l cobalt sulfate and 8 g of crab chitin in powder (Sigma). A control flask (A1) without cobalt sulfate or chitin in the culture medium was performed with the same strain in the same conditions as described. After 48 hours of growth, the medium of each flask was supplemented every 24 hours with 2.2 ml of 100% methanol, 2 ml of 20% casamino acids, 1.5 ml of 20 mg/ml biotin, 200 μl of trace metal solution 1 (see above) and 3.2 ml of 70% glycerol. The induction phase was maintained during a total of 96 hours. Samples of each culture were harvested every 24 hours and chitin deacetylase activity was measured in culture supernatants. Chitin deacetylase activity was measured by radiolabelled method using ³H-glycolchitin and expressed as cpm per ml of culture supernatant. The results are summarized in the table below.

The combination of chitin and cobalt in the culture medium increased CDA activity level up to 2 times in comparison with the control culture without any supplementation.

| | 24 hours | 48 hours | 72 hours | 96 hours |
|---|---|---|---|---|
| Flask | induction | induction | induction | induction |
| A1 | 732 | 755 | 1070 | 1260 |
| A5 | 910 | 1465 | 2640 | 1802 |

### Example 15

### Chitin Deacetylase Expression from pPICZα/CDA Strains

This example illustrates the preparation of recombinant chitin deacetylase using 10 recombinant P. pastoris strains obtained by transformation of GS115 strain with pPICZα/CDA plasmid. Pre-cultures of as GS115/pPICZα-CDA-C(6×HIS) clones 1 to 10 were grown in shake flasks of 100 ml containing 20 ml of YPG medium. Each flask was inoculated with a colony from a fresh agar plate. After 24 h, flasks of 100 ml containing 20 ml of minimal medium (containing only 0.25% of each component by comparison with the same medium used in fermentation) were inoculated with 2 ml of pre-culture. The pH of the medium was adjusted at 4.5 with 16% NH₄OH before autoclaving, and at pH 5.5 after sterilization and cooling. The cultures were grown at 30° C. with orbital shaking at 270 rpm. After 24 h of growth, the induction was started by addition of 111 μl of 100% methanol. The medium was supplemented by addition of 1 ml of 20% casamino acids, 35 μl of 20 mg/ml biotin (20 mg/ml) and 2.5 μl of trace metal solution 1 (see above). After 48 hours of growth, the medium of each flask was supplemented every 24 hours with 110 μl of 100% methanol, 35 μl of 20 mg/ml biotin and 2.5 μl of trace metal solution 1 (see above). The induction phase was maintained during a total of 96 hours. Samples of each culture were harvested every 24 hours and chitin deacetylase activity was measured in culture supernatants. Chitin deacetylase activity was measured by radiolabelled method using ³H-glycolchitin and expressed as cpm per ml of culture supernatant. The results are summarized in the table below.

| | | 72 hours | 96 hours |
|---|---|---|---|
| Strain | Clone number | induction | induction |
| GS115/pPICZα-CDA | 1 | 2 755 | 3 080 |
| GS115/pPICZα-CDA | 2 | 6 865 | 6 935 |
| GS115/pPICZα-CDA | 3 | 3 215 | 4 395 |
| GS115/pPICZα-CDA | 4 | 4 935 | 3 255 |
| GS115/pPICZα-CDA | 5 | 4 295 | 3 005 |
| GS115/pPICZα-CDA | 6 | 6 075 | 3 685 |
| GS115/pPICZα-CDA | 7 | 6 030 | 4 935 |
| GS115/pPICZα-CDA | 8 | 3 610 | 3 035 |
| GS115/pPICZα-CDA | 9 | 4 525 | 3 705 |
| GS115/pPICZα-CDA | 10 | 2 860 | 2 510 |

### Example 16

### Expression of Chitin Deacetylase with N-Terminal Polyhistidine (6×His) Tag

This example illustrates the preparation of recombinant chitin deacetylase using 6 recombinant P. pastoris strains obtained by transformation of GS115 strain with pPIC9/CDA-N(6×His) plasmid. The presence of the (6×His) tag allows the purification of the recombinant chitin deacetylase on metal-chelating resin.

Pre-cultures of GS115/pPIC9-CDA-N(6×HIS) clones 1, 9, 11, 2′, 4′ and 14′ were grown in shake flasks of 100 ml containing 20 ml of YPG medium. Each flask was inoculated with a yeast colony from a fresh agar plate. After 24 h, flasks of 250 ml containing 45 ml of minimal medium (containing only 0.25% of each component by comparison with the same medium used in fermentation) were inoculated with 5 ml of pre-culture. The pH of the medium was adjusted at 4.5 with 16% NH₄OH before autoclaving, and at pH 5.5 after sterilization and cooling. The cultures were grown at 30° C. with orbital shaking at 270 rpm. After 24 h of growth, the induction was started by addition of 250 μl of 100% methanol. The medium was supplemented by addition of 2.5 ml of 20% casamino acids, 87.5 μl of 20 mg/ml biotin, 6.25 μl of trace metal solution 1 (see above) and 840 μl of 70% glycerol. After 48 hours of growth, the medium of each flask was supplemented every 24 hours with 250 μl of 100% methanol, 87.5 μl of 20 mg/ml biotin and 6.25 μl of trace metal solution 1 (see above). The induction phase was maintained during a total of 120 hours. Samples of each culture were harvested every 24 hours and chitin deacetylase activity was measured in culture supernatants. Chitin deacetylase activity was measured by radiolabelled method using ³H-glycolchitin and expressed as cpm per ml of culture supernatant. The results are summarized in the table below.

| | 72 hours | 96 hours | 120 hours |
|---|---|---|---|
| Strains | induction | induction | induction |
| GS115/pPIC9-CDA N(6xHis) 1 | 5 137 | 4 540 | 6 623 |
| GS115/pPIC9-CDA N(6xHis) 9 | 6 380 | 7 773 | 9 197 |
| GS115/pPIC9-CDA N(6xHis) 11 | 1 372 | 3 177 | 3 977 |
| GS115/pPIC9-CDA N(6xHis) 2′ | 6 800 | 8 240 | 7 373 |
| GS115/pPIC9-CDA N(6xHis) 4′ | 6 337 | 8 580 | 9 477 |
| GS115/pPIC9-CDA N(6xHis) 14′ | 6 230 | 8 577 | 9 337 |

Chitin deacetylase expressed from the recombinant clones was recognized by rabbit polyclonal antiserum raised against the native chitin deacetylase from M. rouxii. The presence of a tag(6×His) in the chitin deacetylase expressed from the recombinant clones was identified by immunodetection on western blot with antibodies raised against penta-His extremity (Qiagen). The chitin deacetylase with N-terminal (6×His) tag was purified at homogeneity by IMAC method as described in the example 17.

### Example 17

### Purification of Chitin Deacetylase with N-Terminal Polyhistidine (6×His) Tag

This example illustrates a purification process of chitin deacetylase with N-terminal (6×His) tag according to the present invention. In this example, a supernatant from a 50 ml flask culture was harvested after 120 hours of culture and centrifuged. A sample of 5 ml was applied on a HiTrap affinity column (1 ml, Amersham Pharmacia Biotech) previously loaded with Ni²⁺ ions according to the supplier instructions. After the column was washed, the elution was performed with 5 ml of 20 mM phosphate buffer pH 7.0-7.4 containing 0.5M NaCl and 0.5 M imidazole. Eluted fractions of 1 ml were collected and analyzed for chitin deacetylase activity and purity. The whole chitin deacetylase activity was measured in the first elution fraction (1 ml) and the specific activity was 1.9 10⁶ cpm/mg protein. After electrophoretical analysis on SDS polyacrylamide gel, the enzyme appeared as a single band with an apparent molecular weight of −75 kDa. Western blot analysis with antibodies raised against polyhistidine tag (Penta-His antibody, Qiagen) revealed a single immuno-reactive band corresponding to the same molecular weight.

### Example 18

### Culture of a Recombinant Fungal Strain

The present example illustrates the step according to the present invention wherein a recombinant fungal strain is cultured in a suitable culture medium.

50 ml of AMM medium contained in 250 ml conical flask was prepared by mixing the following compounds: 5 g yeast extract, 6 g NaNO₃, 10 g D-glucose, 26 g l⁻¹ KCl; 76 g l⁻¹ KH₂PO₄; 26 g l⁻¹ MgSO₄. The medium is preferably prepared with deionized water and sterilized, e.g. by autoclaving for 20 min at 120° C. The composition of the culture medium may vary. For example, sucrose, starch, partially hydrolyzed starch (fluitex, glucidex), or other carbon sources, alone or in combination, can be used instead of D-glucose. When the pH of the culture medium was adjusted to 6.5 with 1N NaOH, the medium was autoclaved and then supplemented with a solution comprising trace metals such as 76 μM ZnSO₄; 178 μM H₃BO₃; 25 μM MnCl₂; 18 μM FeSO₄; 7.1 μM CoCl₂; 6.4 μM CuSO₄; 6.2 μM Na₂MoO₄; 174 μM EDTA. This solution was previously sterilized by filtration on a 0.22 μm pore size filter. Subsequently, the medium was inoculated with fresh or frozen spores at an amount of 0.5 10⁵ spores per ml of medium of a recombinant fungal strain prepared according to the present invention. A suitable strain consists of A. oryzae having a pUT970 expression vector and carrying, the gpdA promoter sequence, the cDNA of CDA gene of Mucor rouxii, the Sh ble resistance gene, and the Ssa secretion signal sequence. This strain is also referred to as Aspergillus oryzae pUT970/2-CDA8′ in the present application. The culture was performed with low shaking, preferably 150 rpm, at an ideal temperature between 28° C. and 30° C., during 72 hours to 120 hours. During the culture time, the medium can be further supplemented with nutrients like carbon and nitrogen sources, casamino acids, trace elements and vitamins.

### Example 19

### Measurement of CDA Activity

A sample from a 50 ml culture in 250 ml flask, cultured as described in example 1, was harvested after 96 hours of growth. The sample was filtrated on miracloth filter and the filtrate was concentrated on a centrifugal filter device to reach a final concentration factor of 20 times. Chitin deacetylase activity measured in the sample was 4.0 10³ cpm/ml of supernatant.

It is noted that chitin deacetylase activity is expressed throughout this application in the unit cpm/ml supernatant. Throughout the application, partially O-hydroxyethylated chitin (glycol chitin) radiolabeled in N-acetyl groups is used as substrate. The substrate is synthesized as described in Araki and Ito (1975. Eur. J. Biochem., 55, 71-78). The enzyme activity determination was performed as described in Araki et al. (1975 Eur. J. Biochem., 55, 71-78) and Kafetzopoulos et al. (1993. PNAS 90, 2564-2568). For measurements the same protocol is always applied: 5 μl of this substrate having a total activity of 100.000 cpm is mixed to 20 μl of a sample of the CDA enzyme preparation and to 25 μl of buffer. The use of this protocol allows direct comparison between all obtained CDA activity values as presented in the present application.

It is further noted that another method of enzyme assay comprises determination of acetic acid released during the incubation of chitin deacetylase with chitinous substrates. Therefore, the enzymatic method of Bergmeyer (1974. Methods Enzym. Anal., 1, 112-117) can be used.

### Example 20

### Production of Chitin Deacetylase by a Recombinant Fungal Strain

Aspergillus oryzae pUT970/2-CDA8′ strain was grown in a conical flask of 250 ml containing 50 ml of AMM medium which composition is described hereafter. AMM medium contained 5 g l⁻¹ yeast extract; 6 g l⁻¹ NaNO₃; 30 g l⁻¹ glucidex or sucrose; 7 mM KCl; 11 mM KH₂PO₄; 2 mM MgSO₄. The final volume was adjusted with deionized water. The pH of the medium was adjusted to 6.5 with a solution of 1N NaOH. After autoclaving at 120° C. during 20 min, the medium was supplemented with a solution of trace elements (1 ml per liter of medium) previously sterilized by filtration on 0.22 μm pore size filter. The medium was inoculated with spores of the recombinant A. oryzae strain previously kept at −70° C. in a mixture of 10% (w/v) glycerol and 10% (w/v) skimmed milk. The concentration of the inoculum was 0.5×10⁵ spores per ml of medium. The culture was grown at 30° C., shaken at 150 rpm during 120 hours.

Samples were harvested and analyzed every 24 hours. Culture fluid and mycelium were separated by filtration and the mycelium was crushed in TE buffer containing PMSF. The extract was then centrifuged and the supernatant corresponding to the soluble intracellular fraction was recovered. Chitin deacetylase activity (radiometric assay using radiolabelled glycolchitin) was measured in the culture supernatant and in the soluble intracellular fraction. Both supernatants were previously concentrated 20 times on centrifugal filter devices.

The maximum of chitin deacetylase activity was obtained in the culture supernatant harvested after 96 hours of culture in AMM medium containing 30 g l⁻¹ sucrose. It was estimated to 4.02×10³ cpm/ml of medium. Comparatively, the level of chitin deacetylase expression in the culture grown in AMM medium containing 30 g l⁻¹ glucidex was 2.74×10³ cpm/ml of supernatant harvested at the same culture time. In both cases, chitin deacetylase activity was also measured in the soluble intracellular fraction but the level of enzyme activity was lower: respectively 1.02×10³ cpm/ml of medium, and 0.58×10³ cpm/ml of medium.

### Example 21

### Production of Chitin Deacetylase by a Recombinant Fungal Strain

Aspergillus oryzae pUT970/2-CDA8′ strain was grown in a conical flask of 250 ml containing 50 ml of AMM medium which composition is described hereafter. AMM medium contained 5 g l⁻¹ yeast extract; 6 g l⁻¹ NaNO₃; 30 g l⁻¹ glucidex or sucrose; 7 mM KCl; 11 mM KH₂PO₄; 2 mM MgSO₄. The final volume was adjusted with deionized water. The pH of the medium was adjusted to 6.5 with a solution of 1N NaOH. After autoclaving at 120° C. during 20 min, the medium was supplemented with a solution of trace elements (1 ml per liter of medium) previously sterilized by filtration on 0.22 μm pore size filter.

The medium was inoculated with 0.5 10⁵ spores per ml of medium of the recombinant A. oryzae strain previously kept at −70° C. in a mixture of 10% (w/v) glycerol and 10% (w/v) skim-milk The culture was grown at 30° C., shaken at 150 rpm during 96 hours. After 36 hours of incubation, the medium was supplemented with 10 g l⁻¹ or 30 g l⁻¹ glucidex or sucrose. This addition of nutrients was repeated every 12 hours until the end of the culture. After 96 hours of culture, the supernatant was clarified by filtration and centrifugation. The chitin deacetylase activity was measured in the supernatant previously concentrated 20 times.

Comparatively, the level of chitin deacetylase activity was 2 times higher when the culture medium was supplemented with 10 g l⁻¹ than with 30 g l⁻¹ of the carbon source.

| | CDA activity (10³ cpm/ml) |
|---|---|
| Sucrose medium suppl. with 1% sucrose | 2.86 |
| Sucrose medium suppl. with 3% sucrose | 1.34 |
| Glucidex medium suppl. with 1% glucidex | 3.11 |
| Glucidex medium suppl. with 3% glucidex | 1.37 |

Both carbon sources, glucidex and sucrose, provide similar results of chitin deacetylase expression. Using a supplementation of the medium with 1% of carbon source provides better results of chitin deacetylase expression than with a supplementation of 3%.

### Example 22

### Purification of CDA from a Fungal Culture

Supernatant resulting from a 100 ml culture in AMM medium with 30 g l⁻¹ sucrose at pH 6.5 was purified. After 96 hours of culture, the culture fluid was collected by filtration. It was then dialyzed against 5 mM sodium succinate buffer/pH 5.5 (buffer A). The used strain consisted of A. oryzae having a pUT970 expression vector and carrying, the gpdA promoter sequence, the cDNA of CDA gene of Mucor rouxii, the Shble resistance gene, and the Ssa secretion signal sequence. 30 ml of supernatant was applied onto a column and CDA activity in the crude sample comprised 7.3×10⁵ cpm/ml. The sample was applied onto a Q Sepharose Fast Flow column (30 ml) previously equilibrated in buffer A. The column was washed with buffer A and the elution was performed with a step gradient of buffer A/NaCl corresponding to a gradient of conductivity from 0 ms/cm to 20 ms/cm. 5 ml fractions were collected and analyzed. All fractions (6×5 ml) containing chitin deacetylase activity were pooled. Total chitin deacetylase activity was 1.7 10⁵ cpm (radiometric assay), corresponding to an enzyme activity of 5.6×10³ cpm/ml of purified chitin deacetylase.

What is claimed is:

1. A recombinant fungal strain, capable of expressing chitin deacetylase, comprising an expression vector that contains a suitable promoter, a nucleic acid molecule encoding chitin deacetylase, a selectable marker sequence, a secretion signal sequence, and a transcription terminator.

2. The recombinant fungal strain according to claim 1, wherein said fungal strain is a filamentous fungus.

3. The recombinant fungal strain according to claim 1, wherein said fungal strain is selected from the group consisting of the genera Aspergillus, Emericella, Trichoderma, Achlya, Neurospora, Phanerochaete, Tolyplocadium and Penicillium.

4. The recombinant fungal strain according to claim 1, comprising an expression vector that contains a nucleic acid molecule encoding chitin deacetylase obtained from Mucor rouxii.

5. The recombinant fungal strain according to claim 1, comprising an expression vector wherein the promoter is a constitutive or an inducible promoter.

6. The recombinant fungal strain according to claim 1 comprising an expression vector, wherein the selectable marker gene is an auxotrophic marker gene or a dominant marker gene.

7. The recombinant fungal strain according to claim 1, comprising an expression vector wherein the nucleic acid molecule encoding chitin deacetylase is expressed as a translational fusion to the C-terminus of the selection marker protein such that chitin deacetylase is expressed as a C-terminal fusion protein.

8. The recombinant fungal strain according to claim 1, comprising an expression vector that further comprises additional nucleotide sequences provided at the 5′ and/or 3′ terminal end of the nucleic acid molecule encoding chitin deacetylase.

9. The recombinant fungal strain that has all of the identifying characteristics of deposit No IHEM 20351, deposited at the BCCM-IHEM, biomedical fungi and yeasts collection, Scientific Institute of Public Health, Louis Pasteur, Brussels Belgium on Feb. 10, 2004.

10. A method for producing chitin deacetylase by a recombinant fungal strain comprising the steps of:
constructing a recombinant fungal strain capable of expressing chitin deacetylase by recombinant DNA techniques,
preparing a culture comprising spores of said recombinant fungal strain,
inoculating a suitable amount of spores of said recombinant fungal strain in a suitable medium and incubating said recombinant fungal strain in said medium for a suitable period of time,
feeding for a suitable period of time said incubated recombinant fungal strain with a suitable substrate which controls proliferation of the fungal strain,
clarifying the medium such that fungal mycelium is removed from the medium and the supernatant of the medium which comprises chitin deacetylase is retained, and
isolating said chitin deacetylase from said supernatant by means of chromatographic techniques.

11. The method according to claim 10, wherein a recombinant fungal strain capable of expressing chitin deacetylase is constructed which comprises an expression vector that contains a promoter, a nucleic acid molecule encoding chitin deacetylase, a selectable marker sequence, a secretion signal sequence, and a transcription terminator.

12. The method according to claim 10, wherein said method comprises construction of a recombinant fungal strain capable of expressing chitin deacetylase, wherein said fungal strain is a filamentous fungus.

13. The method according to claim 10, wherein said method comprises construction of a recombinant fungal strain comprising an expression vector that comprises a nucleic acid molecule encoding chitin deacetylase obtained from Mucor rouxii.

14. The method according to claim 10, wherein said method comprises construction of a recombinant fungal strain comprising an expression vector wherein the selectable marker gene is an auxotrophic marker gene or a dominant marker gene.

15. The method according to claim 10, wherein said method comprises construction of a recombinant fungal strain comprising an expression vector wherein the promoter is a constitutive or an inducible promoter.

16. The method according to claim 10, wherein said method comprises construction of a recombinant fungal strain comprising an expression vector wherein the nucleic acid molecule encoding chitin deacetylase is expressed as a translational fusion to the C-terminus of the selection marker protein such that chitin deacetylase is expressed as a C-terminal fusion protein.

17. The method according to claim 10, wherein said method comprises construction of a recombinant fungal strain comprising an expression vector that further comprises additional nucleotide sequences provided at the 5′ end and/or at the 3′ end of the nucleic acid molecule encoding chitin deacetylase.

18. The method according to claim 10, comprising the step of inoculating an amount of spores of said recombinant fungal strain comprised between 0.5×10⁵ and 1.0×10⁸ spores per ml medium.

19. The method according to claim 10, comprising clarifying said medium by filtration, centrifugation and/or micro-filtration such that fungal mycelium is removed from the medium.

20. A purified recombinant chitin deacetylase obtainable by a method according to claim 10.

21. A recombinant yeast strain, capable of expressing chitin deacetylase, comprising an expression vector that contains a nucleic acid molecule encoding chitin deacetylase, a suitable promoter and a transcription terminator.

22. The recombinant yeast strain according to claim 21, wherein said yeast strain is a methylotrophic yeast.

23. The recombinant yeast strain according to claim 21, wherein said yeast strain is selected from the genus Pichia or Hansenula.

24. The recombinant yeast strain according to claim 21, wherein said yeast strain is a Pichia pastoris or a Pichia methanolica strain.

25. The recombinant yeast strain according to claim 21, wherein said Pichia pastoris recombinant yeast strain comprises an expression vector selected from the group consisting of pFLD1α, pPIC9, pHIL-S1, pPIC9K, pPIC6α, pPICZα-E and pPICZα and wherein said Pichia methanolica recombinant yeast strain comprises a pMETα expression vector.

26. The recombinant yeast strain according to claim 21, comprising an expression vector that contains a nucleic acid molecule encoding chitin deacetylase obtained from Mucor rouxii.

27. The recombinant yeast strain according to claim 21, comprising an expression vector that contains an inducible promoter.

28. The recombinant yeast strain according to claim 27, comprising an expression vector that contains a methanol-inducible promoter.

29. The recombinant yeast strain according to claim 27, comprising an expression vector that contains a Pichia pastoris alcohol oxidase 1 (AOX1) promoter or a Pichia methanolica alcohol utilization gene 1 (AUG1) promoter.

30. The recombinant yeast strain according to claim 21, comprising an expression vector that further comprises a selectable marker gene selected from the group consisting of HIS4, ADE2, ADE1, ARG4, URA3 and antibiotic resistance genes.

31. The recombinant yeast strain according to claim 21, comprising an expression vector that further comprises a secretion signal sequence to direct the transport of the chitin deacetylase protein to the extracellular medium.

32. The recombinant yeast strain according to claim 31, wherein said secretion signal sequence is the nucleotide sequence coding for the Saccharomyces cerevisiae α-factor prepro peptide or the nucleotide sequence coding for the Pichia pastoris alkaline phosphatase signal peptide or the nucleotide sequence coding for the PHA-E signal peptide from the plant lectin Phaseolus vulgaris agglutinin.

33. The recombinant yeast strain according to claim 21, comprising an expression vector that further comprises additional nucleotide sequences provided at the 5′ and/or 3′ terminal end of the nucleic acid molecule encoding chitin deacetylase.

34. A recombinant yeast strain that has all of the identifying characteristics of accession number MUCL 44353, deposited at the BCCM-MUCL fungi and yeast Collection in the Scientific Institute of Public Health, Louis Pasteur, Brussels, Belgium on Jan. 24, 2003.

35. A recombinant yeast multicopy strain capable of expressing chitin deacetylase, comprising more than one copy of the nucleic acid molecule encoding chitin deacetylase from Mucor rouxii.

36. A method for producing chitin deacetylase by a recombinant yeast strain comprising the steps of:
constructing a recombinant yeast strain capable of expressing chitin deacetylase according to claim 21 by recombinant DNA techniques,
preparing a pre-culture comprising said recombinant yeast strain,
inoculating a suitable amount of said pre-cultured recombinant yeast strain in a suitable fermentation medium and incubating said recombinant yeast strain in said fermentation medium for a suitable period,
feeding for a suitable period of time said incubated recombinant yeast strain with a suitable substrate which controls proliferation of the yeast,
supplementing for a suitable period of time the medium of the incubated recombinant yeast strain with an inducer, capable of stimulating the transcription and translation of the gene encoding chitin deacetylase,
clarifying the medium such that yeast cells are removed from the medium and the supernatant of the medium which comprises chitin deacetylase is retained, and
isolating said chitin deacetylase from said supernatant by chromatographic techniques.

37. The method according to claim 36, comprising inoculating between 5 and 10% of the initial fermentation volume of said pre-cultured recombinant yeast strain in a fermentation medium.

38. The method according to claim 36, comprising supplementing said fermentation medium with a component selected from the group consisting of surfactants, metallic ions, an inducer of the expression of chitin-related enzymes and any combinations thereof.

39. The method according to claim 36, comprising feeding the incubated recombinant yeast at a rate of 5 to 10 ml of substrate per hour per liter of fermentation medium.

40. The method according to claim 36, comprising supplementing the medium of the incubated recombinant yeast with an inducer being alcohol.

41. The method according to claim 36, comprising clarifying said fermentation medium by centrifugation and micro-filtration such that yeast cells are removed from the medium.

42. A purified recombinant chitin deacetylase obtainable by a method according to claim 36.

43. The method according to claim 40, wherein the alcohol is methanol.
